(19) United States
(12) Patent Application Publication
(10) Pub. No.: US 2024/0416130 A1
(43) Pub. Date: Dec. 19, 2024

(54) NEUROSTIMULATION TITRATION UTILIZING T-WAVE ALTERNANS

(71) Applicant: LivaNova USA, Inc., Houston, TX (US)

(72) Inventors: Imad Libbus, St. Paul, MN (US); Bruce H. KenKnight, Maple Grove, MN (US)

(73) Assignee: LivaNova USA, Inc., Houston, TX (US)

(21) Appl. No.: 18/821,703

(22) Filed: Aug. 30, 2024

Related U.S. Application Data
(63) Continuation of application No. 17/129,467, filed on Dec. 21, 2020, now Pat. No. 12,076,566.
Application No. 17/129,467 is a continuation of application No. 16/159,415, filed on Oct. 12, 2018, now Pat. No. 10,870,005.
Application No. 16/159,415 is a continuation of application No. 14/938,641, filed on Nov. 11, 2015, now Pat. No. 10,099,055.
(60) Provisional application No. 62/078,600, filed on Nov. 12, 2014.

(51) Int. Cl. A61N 1/36; A61B 5/349; A61B 5/355; A61N 1/05; A61N 1/37; A61N 1/372; G16H 20/30; G16H 20/40; G16H 40/20; G16H 40/63; G16Z 99/00

(52) U.S. Cl. CPC A61N 1/36139; A61B 5/349; A61B 5/355; A61N 1/0551; A61N 1/36114; A61N 1/36132; A61N 1/3615; A61N 1/36167; A61N 1/3702; A61N 1/37223; A61N 1/37235; G16H 20/30; G16H 20/40; G16H 40/20; G16H 40/63; G16Z 99/00

(57) ABSTRACT

Systems and methods are provided for delivering neurostimulation therapies to patients. A titration process is used to gradually increase the stimulation intensity to a desired therapeutic level until a target T-wave alternans change from a baseline T-wave alternans is achieved.

### CROSS-REFERENCE TO RELATED APPLICATIONS

This application is a Continuation of U.S. patent application Ser. No. 17/129,467, filed Dec. 21, 2020, which is a Continuation of U.S. patent application Ser. No. 16/159,415, filed Oct. 12, 2018, issued as U.S. Pat. No. 10,870,005, which is a Continuation of U.S. patent application Ser. No. 14/938,641, filed Nov. 11, 2015, issued as U.S. Pat. No. 10,099,055, which claims priority to U.S. Provisional Application No. 62/078,600, filed on Nov. 12, 2014, each of which are incorporated herein by reference in their entireties.

### FIELD

This application relates to neuromodulation and, more specifically, to improved systems and methods for titrating stimulation therapies.

### BACKGROUND

Chronic heart failure (CHF) and other forms of chronic cardiac dysfunction (CCD) may be related to an autonomic imbalance of the sympathetic and parasympathetic nervous systems that, if left untreated, can lead to cardiac arrhythmogenesis, progressively worsening cardiac function and eventual patient death. CHF is pathologically characterized by an elevated neuroexitatory state and is accompanied by physiological indications of impaired arterial and cardiopulmonary baroreflex function with reduced vagal activity.

CHF triggers compensatory activations of the sympathoadrenal (sympathetic) nervous system and the renin-angiotensin-aldosterone hormonal system, which initially helps to compensate for deteriorating heart-pumping function, yet, over time, can promote progressive left ventricular dysfunction and deleterious cardiac remodeling. Patients suffering from CHF are at increased risk of tachyarrhythmias, such as atrial fibrillation (AF), ventricular tachyarrhythmias (ventricular tachycardia (VT) and ventricular fibrillation (VF)), and atrial flutter, particularly when the underlying morbidity is a form of coronary artery disease, cardiomyopathy, mitral valve prolapse, or other valvular heart disease. Sympathoadrenal activation also significantly increases the risk and severity of tachyarrhythmias due to neuronal action of the sympathetic nerve fibers in, on, or around the heart and through the release of epinephrine (adrenaline), which can exacerbate an already-elevated heart rate.

The standard of care for managing CCD in general continues to evolve. For instance, new therapeutic approaches that employ electrical stimulation of neural structures that directly address the underlying cardiac autonomic nervous system imbalance and dysregulation have been proposed. In one form, controlled stimulation of the cervical vagus nerve beneficially modulates cardiovascular regulatory function. Vagus nerve stimulation (VNS) has been used for the clinical treatment of drug-refractory epilepsy and depression, and more recently has been proposed as a therapeutic treatment of heart conditions such as CHF. For instance, VNS has been demonstrated in canine studies as efficacious in simulated treatment of AF and heart failure, such as described in Zhang et al., “Chronic Vagus Nerve Stimulation Improves Autonomic Control and Attenuates Systemic Inflammation and Heart Failure Progression in a Canine High-Rate Pacing Model,” Circ Heart Fail 2009, 2, pp. 692-699 (Sep. 22, 2009), the disclosure of which is incorporated by reference. The results of a multi-center open-label phase II study in which chronic VNS was utilized for CHF patients with severe systolic dysfunction is described in De Ferrari et al., “Chronic Vagus Nerve Stimulation: A New and Promising Therapeutic Approach for Chronic Heart Failure,” European Heart Journal, 32, pp. 847-855 (Oct. 28, 2010).

VNS therapy commonly requires implantation of a neurostimulator, a surgical procedure requiring several weeks of recovery before the neurostimulator can be activated and a patient can start receiving VNS therapy. Even after the recovery and activation of the neurostimulator, a full therapeutic dose of VNS is not immediately delivered to the patient to avoid causing significant patient discomfort and other undesirable side effects. Instead, to allow the patient to adjust to the VNS therapy, a titration process is utilized in which the intensity is gradually increased over a period of time under a control of a physician, with the patient given time between successive increases in VNS therapy intensity to adapt to the new intensity. As stimulation is chronically applied at each new intensity level, the patient's tolerance threshold, or tolerance zone boundary, gradually increases, allowing for an increase in intensity during subsequent titration sessions. The titration process can take significantly longer in practice because the increase in intensity is generally performed by a physician or other healthcare provider, and thus, for every step in the titration process to take place, the patient has to visit the provider's office to have the titration performed. Scheduling conflicts in the provider's office may increase the time between titration sessions, thereby extending the overall titration process, during which the patient in need of VNS does not receive the VNS at the full therapeutic intensity.

For patients receiving VNS therapy for the treatment of epilepsy, a titration process that continues over an extended period of time, such as six to twelve months, may be somewhat acceptable because the patient's health condition typically would not worsen in that period of time. However, for patients being treated for other health conditions, such as CHF, the patient's condition may degrade rapidly if left untreated. As a result, there is a much greater urgency to completing the VNS titration process when treating a patient with a time-sensitive condition, such as CHF.

Accordingly, a need remains for an approach to efficiently titrate neurostimulation therapy for treating chronic cardiac dysfunction and other conditions.

### SUMMARY

Systems and methods are provided for delivering neurostimulation therapies to patients. A titration process is used to gradually increase the stimulation intensity to a desired therapeutic level until a target T-wave alternans change from a baseline T-wave alternans is achieved.

Still other embodiments of the present invention will become readily apparent to those skilled in the art from the following detailed description, wherein are described embodiments by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF DRAWINGS

FIG. 1 is a front anatomical diagram showing, by way of example, placement of an implantable vagus stimulation device in a male patient, in accordance with one embodiment.

FIGS. 2A and 2B are diagrams respectively showing the implantable neurostimulator and the simulation therapy lead of FIG. 1.

FIG. 3 is a diagram showing an external programmer for use with the implantable neurostimulator of FIG. 1.

FIG. 4 is a diagram showing electrodes provided as on the stimulation therapy lead of FIG. 2 in place on a vagus nerve in situ.

FIG. 5 is a graph showing, by way of example, the relationship between the targeted therapeutic efficacy and the extent of potential side effects resulting from use of the implantable neurostimulator of FIG. 1.

FIG. 6 is a graph showing, by way of example, the optimal duty cycle range based on the intersection depicted in FIG. 5.

FIG. 7 is a timing diagram showing, by way of example, a stimulation cycle and an inhibition cycle of VNS as provided by implantable neurostimulator of FIG. 1.

FIGS. 8A-8C are illustrative charts reflecting a heart rate response to gradually increased stimulation intensity at different frequencies.

FIG. 9 illustrates a method for delivering vagus nerve stimulation therapy.

FIG. 10 illustrates a titration process in accordance with embodiments of the present invention.

FIGS. 11A-11B are block diagrams of neurostimulation systems in accordance with embodiments of the present invention.

FIG. 12 illustrates a titration process with variable titration parameters in accordance with embodiments of the present invention.

FIG. 13 is a scatter plot illustrating stimulation output current and corresponding T-wave alternans changes from baseline.

FIGS. 14A-14B are illustrations of a method of assessing T-wave alternans in accordance with embodiments of the present invention.

FIGS. 15A-15B are plots illustrating heart rate turbulence response for patients receiving VNS therapy in accordance with embodiments of the present invention.

FIG. 16 is a bar chart illustrating T-wave alternan magnitude levels for patients receiving different stimulation levels in accordance with embodiments of the present invention.

FIG. 17 is a bar chart illustrating heart rate turbulence slope levels for patients receiving different stimulation levels in accordance with embodiments of the present invention.

### DETAILED DESCRIPTION

CHF and other cardiovascular diseases cause derangement of autonomic control of the cardiovascular system, favoring increased sympathetic and decreased parasympathetic central outflow. These changes are accompanied by elevation of basal heart rate arising from chronic sympathetic hyperactivation along the neurocardiac axis.

The vagus nerve is a diverse nerve trunk that contains both sympathetic and parasympathetic fibers, and both afferent and efferent fibers. These fibers have different diameters and myelination, and subsequently have different activation thresholds. This results in a graded response as intensity is increased. Low intensity stimulation results in a progressively greater tachycardia, which then diminishes and is replaced with a progressively greater bradycardia response as intensity is further increased. Peripheral neurostimulation therapies that target the fluctuations of the autonomic nervous system have been shown to improve clinical outcomes in some patients. Specifically, autonomic regulation therapy results in simultaneous creation and propagation of efferent and afferent action potentials within nerve fibers comprising the cervical vagus nerve. The therapy directly improves autonomic balance by engaging both medullary and cardiovascular reflex control components of the autonomic nervous system. Upon stimulation of the cervical vagus nerve, action potentials propagate away from the stimulation site in two directions, efferently toward the heart and afferently toward the brain. Efferent action potentials influence the intrinsic cardiac nervous system and the heart and other organ systems, while afferent action potentials influence central elements of the nervous system.

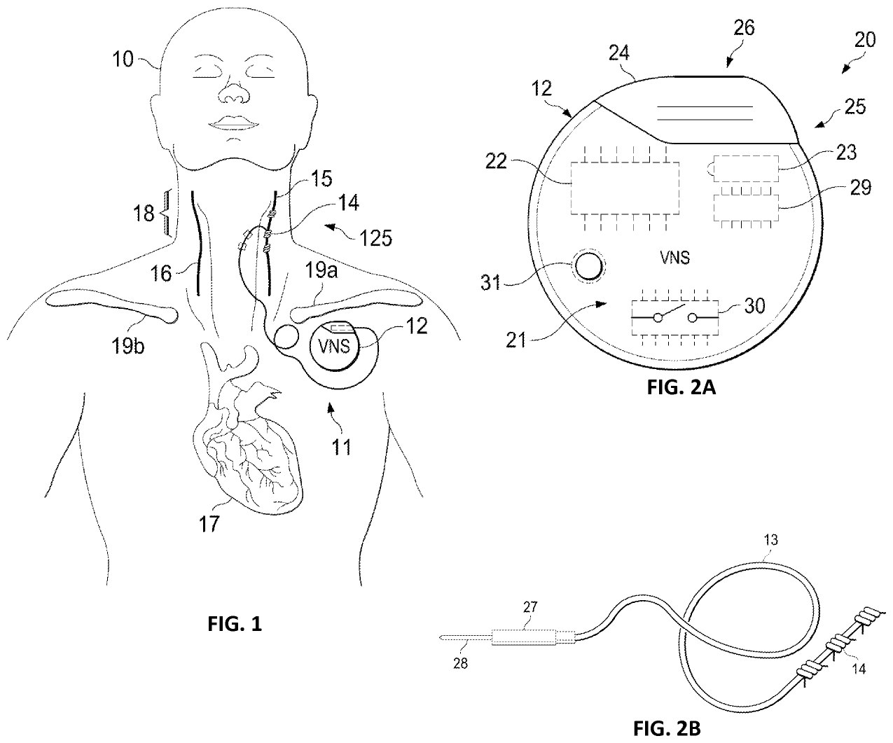

An implantable vagus nerve stimulator, such as used to treat drug-refractory epilepsy and depression, can be adapted for use in managing chronic cardiac dysfunction (CCD) through therapeutic bi-directional vagus nerve stimulation. FIG. 1 is a front anatomical diagram showing, by way of example, placement of an implantable medical device (e.g., a vagus nerve stimulation (VNS) system 11, as shown in FIG. 1) in a male patient 10, in accordance with embodiments of the present invention. The VNS provided through the stimulation system 11 operates under several mechanisms of action. These mechanisms include increasing parasympathetic outflow and inhibiting sympathetic effects by inhibiting norepinephrine release and adrenergic receptor activation. More importantly, VNS triggers the release of the endogenous neurotransmitter acetylcholine and other peptidergic substances into the synaptic cleft, which has several beneficial anti-arrhythmic, anti-apoptotic, and anti-inflammatory effects as well as beneficial effects at the level of the central nervous system.

The implantable vagus stimulation system 11 comprises an implantable neurostimulator or pulse generator 12 and a stimulating nerve electrode assembly 125. The stimulating nerve electrode assembly 125, preferably comprising at least an electrode pair, is conductively connected to the distal end of an insulated, electrically conductive lead assembly 13 and electrodes 14. The electrodes 14 may be provided in a variety of forms, such as, e.g., helical electrodes, probe electrodes, cuff electrodes, as well as other types of electrodes.

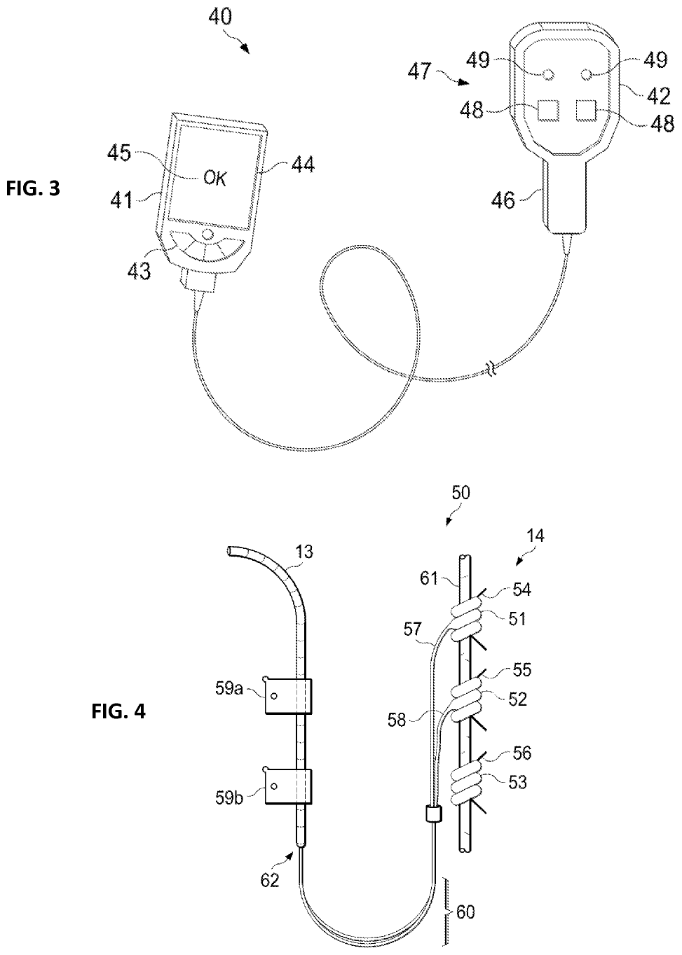

The implantable vagus stimulation system 11 can be remotely accessed following implant through an external programmer, such as the programmer 40 shown in FIG. 3 and described in further detail below. The programmer 40 can be used by healthcare professionals to check and program the neurostimulator 12 after implantation in the patient 10 and to adjust stimulation parameters during the initial stimulation titration process. In some embodiments, an external magnet may provide basic controls, such as described in commonly assigned U.S. Pat. No. 8,600,505, entitled “Implantable Device For Facilitating Control Of Electrical Stimulation Of Cervical Vagus Nerves For Treatment Of Chronic Cardiac Dysfunction,” the disclosure of which is incorporated by reference. For further example, an electromagnetic controller may enable the patient 10 or healthcare professional to interact with the implanted neurostimulator 12 to exercise increased control over therapy delivery and suspension, such as described in commonly-assigned U.S. Pat. No. 8,571,654, entitled “Vagus Nerve Neurostimulator With Multiple Patient-Selectable Modes For Treating Chronic Cardiac Dysfunction,” the disclosure of which is incorporated by reference. For further example, an external programmer may communicate with the neurostimulation system 11 via other wired or wireless communication methods, such as, e.g., wireless RF transmission. Together, the implantable vagus stimulation system 11 and one or more of the external components form a VNS therapeutic delivery system.

The neurostimulator 12 is typically implanted in the patient's right or left pectoral region generally on the same side (ipsilateral) as the vagus nerve 15, 16 to be stimulated, although other neurostimulator-vagus nerve configurations, including contra-lateral and bi-lateral are possible. A vagus nerve typically comprises two branches that extend from the brain stem respectively down the left side and right side of the patient, as seen in FIG. 1. The electrodes 14 are generally implanted on the vagus nerve 15, 16 about halfway between the clavicle 19a-b and the mastoid process. The electrodes may be implanted on either the left or right side. The lead assembly 13 and electrodes 14 are implanted by first exposing the carotid sheath and chosen branch of the vagus nerve 15, 16 through a latero-cervical incision (perpendicular to the long axis of the spine) on the ipsilateral side of the patient's neck 18. The helical electrodes 14 are then placed onto the exposed nerve sheath and tethered. A subcutaneous tunnel is formed between the respective implantation sites of the neurostimulator 12 and helical electrodes 14, through which the lead assembly 13 is guided to the neurostimulator 12 and securely connected.

In one embodiment, the neural stimulation is provided as a low level maintenance dose independent of cardiac cycle. The stimulation system 11 bi-directionally stimulates either the left vagus nerve 15 or the right vagus nerve 16. However, it is contemplated that multiple electrodes 14 and multiple leads 13 could be utilized to stimulate simultaneously, alternatively or in other various combinations. Stimulation may be through multimodal application of continuously-cycling, intermittent and periodic electrical stimuli, which are parametrically defined through stored stimulation parameters and timing cycles. Both sympathetic and parasympathetic nerve fibers in the vagosympathetic complex are stimulated. A study of the relationship between cardiac autonomic nerve activity and blood pressure changes in ambulatory dogs is described in J. Hellyer et al., “Autonomic Nerve Activity and Blood Pressure in Ambulatory Dogs,” Heart Rhythm, Vol. 11(2), pp. 307-313 (February 2014). Generally, cervical vagus nerve stimulation results in propagation of action potentials from the site of stimulation in a bi-directional manner. The application of bi-directional propagation in both afferent and efferent directions of action potentials within neuronal fibers comprising the cervical vagus nerve improves cardiac autonomic balance. Afferent action potentials propagate toward the parasympathetic nervous system's origin in the medulla in the nucleus ambiguus, nucleus tractus solitarius, and the dorsal motor nucleus, as well as towards the sympathetic nervous system's origin in the intermediolateral cell column of the spinal cord. Efferent action potentials propagate toward the heart 17 to activate the components of the heart's intrinsic nervous system. Either the left or right vagus nerve 15, 16 can be stimulated by the stimulation system 11. The right vagus nerve 16 has a moderately lower (approximately 30%) stimulation threshold than the left vagus nerve 15 for heart rate effects at the same stimulation frequency and pulse width.

The VNS therapy is delivered autonomously to the patient's vagus nerve 15, 16 through three implanted components that include a neurostimulator 12, lead assembly 13, and electrodes 14. FIGS. 2A and 2B are diagrams respectively showing the implantable neurostimulator 12 and the stimulation lead assembly 13 of FIG. 1. In one embodiment, the neurostimulator 12 can be adapted from a VNS THERAPY DEMIPULSE Model 103 or ASPIRESR Model 106 pulse generator, manufactured and sold by LivaNova PLC, Houston, Texas, although other manufactures and types of implantable VNS neurostimulators could also be used. The stimulation lead assembly 13 and electrodes 14 are generally fabricated as a combined assembly and can be adapted from a Model 302 lead, PERENNIADURA Model 303 lead, or PERENNIAFLEX Model 304 lead, also manufactured and sold by LivaNova PLC, in three sizes based, for example, on a helical electrode inner diameter, although other manufactures and types of single-pin receptacle-compatible therapy leads and electrodes could also be used.

Referring first to FIG. 2A, the system 20 may be configured to provide multimodal vagus nerve stimulation. In a maintenance mode, the neurostimulator 12 is parametrically programmed to deliver continuously-cycling, intermittent and periodic ON-OFF cycles of VNS. Such delivery produces action potentials in the underlying nerves that propagate bi-directionally, both afferently and efferently.

The neurostimulator 12 includes an electrical pulse generator that is tuned to improve autonomic regulatory function by triggering action potentials that propagate both afferently and efferently within the vagus nerve 15, 16. The neurostimulator 12 is enclosed in a hermetically sealed housing 21 constructed of a biocompatible material, such as titanium. The housing 21 contains electronic circuitry 22 powered by a battery 23, such as a lithium carbon monofluoride primary battery or a rechargeable secondary cell battery. The electronic circuitry 22 may be implemented using complementary metal oxide semiconductor integrated circuits that include a microprocessor controller that executes a control program according to stored stimulation parameters and timing cycles; a voltage regulator that regulates system power; logic and control circuitry, including a recordable memory 29 within which the stimulation parameters are stored, that controls overall pulse generator function, receives and implements programming commands from the external programmer, or other external source, collects and stores telemetry information, processes sensory input, and controls scheduled and sensory-based therapy outputs; a transceiver that remotely communicates with the external programmer using radio frequency signals; an antenna, which receives programming instructions and transmits the telemetry information to the external programmer; and a reed switch 30 that provides remote access to the operation of the neurostimulator 12 using an external programmer, a simple patient magnet, or an electromagnetic controller. The recordable memory 29 can include both volatile (dynamic) and non-volatile/persistent (static) forms of memory, within which the stimulation parameters and timing cycles can be stored. Other electronic circuitry and components are possible.

The neurostimulator 12 includes a header 24 to securely receive and connect to the lead assembly 13. In one embodiment, the header 24 encloses a receptacle 25 into which a single pin for the lead assembly 13 can be received, although two or more receptacles could also be provided, along with the corresponding electronic circuitry 22. The header 24 internally includes a lead connector block (not shown), a setscrew, and a spring contact (not shown) that electrically connects to the lead ring, thus completing the electrical circuit 26.

In some embodiments, the housing 21 may also contain a heart rate sensor 31 that is electrically interfaced with the logic and control circuitry, which receives the patient's sensed heart rate as sensory inputs. The heart rate sensor 31 monitors heart rate using an electrocardiogram (ECG)-type electrode. Through the electrode, the patient's heart beat can be sensed by detecting ventricular depolarization. In a further embodiment, a plurality of electrodes can be used to sense voltage differentials between electrode pairs, which can undergo signal processing for cardiac physiological measures, for instance, detection of the P-wave, QRS complex, and T-wave. The heart rate sensor 31 provides the sensed heart rate to the control and logic circuitry as sensory inputs that can be used to determine the onset or presence of arrhythmias, particularly VT, and/or to monitor and record changes in the patient's heart rate over time or in response to applied stimulation signals.

Referring next to FIG. 2B, the lead assembly 13 delivers an electrical signal from the neurostimulator 12 to the vagus nerve 15, 16 via the electrodes 14. On a proximal end, the lead assembly 13 has a lead connector 27 that transitions an insulated electrical lead body to a metal connector pin 28 and metal connector ring. During implantation, the connector pin 28 is guided through the receptacle 25 into the header 24 and securely fastened in place using a setscrew (not shown) that engages the connector pin 28 to electrically couple one electrode of the lead assembly 13 to the neurostimulator 12 while the spring contact makes electrical contact to the ring connected to the other electrode. On a distal end, the lead assembly 13 terminates with the electrode 14, which bifurcates into a pair of anodic and cathodic electrodes 62 (as further described infra with reference to FIG. 4). In one embodiment, the lead connector 27 is manufactured using silicone and the connector pin 28 and ring are made of stainless steel, although other suitable materials could be used, as well. The insulated lead body 13 utilizes a silicone-insulated alloy conductor material.

In some embodiments, the electrodes 14 are helical and placed around the cervical vagus nerve 15, 16 at the location below where the superior and inferior cardiac branches separate from the cervical vagus nerve. In alternative embodiments, the helical electrodes may be placed at a location above where one or both of the superior and inferior cardiac branches separate from the cervical vagus nerve. In one embodiment, the helical electrodes 14 are positioned around the patient's vagus nerve oriented with the end of the helical electrodes 14 facing the patient's head. In an alternate embodiment, the helical electrodes 14 are positioned around the patient's vagus nerve 15, 16 oriented with the end of the helical electrodes 14 facing the patient's heart 17. At the distal end, the insulated electrical lead body 13 is bifurcated into a pair of lead bodies that are connected to a pair of electrodes. The polarity of the electrodes could be configured into a proximal anode and a distal cathode, or a proximal cathode and a distal anode.

The neurostimulator 12 may be interrogated prior to implantation and throughout the therapeutic period with a healthcare provider-operable control system comprising an external programmer and programming wand (shown in FIG. 3) for checking proper operation, downloading recorded data, diagnosing problems, and programming operational parameters, such as described in commonly-assigned U.S. Pat. Nos. 8,600,505 and 8,571,654, cited supra. FIG. 3 is a diagram showing an external programmer 40 for use with the implantable neurostimulator 12 of FIG. 1. The external programmer 40 includes a healthcare provider operable programming computer 41 and a programming wand 42. Generally, use of the external programmer is restricted to healthcare providers, while more limited manual control is provided to the patient through “magnet mode.”

In one embodiment, the external programmer 40 executes application software 45 specifically designed to interrogate the neurostimulator 12. The programming computer 41 interfaces to the programming wand 42 through a wired or wireless data connection. The programming wand 42 can be adapted from a Model 201 Programming Wand, manufactured and sold by LivaNova PLC, and the application software 45 can be adapted from the Model 250 Programming Software suite, licensed by LivaNova PLC. Other configurations and combinations of external programmer 40, programming wand 42 and application software 45 are possible.

The programming computer 41 can be implemented using a general purpose programmable computer and can be a personal computer, laptop computer, ultrabook computer, netbook computer, handheld computer, tablet computer, smart phone, or other form of computational device. In one embodiment, the programming computer is a tablet computer that may operate under the iOS operating system from Apple Inc., such as the iPad from Apple Inc., or may operate under the Android operating system from Google Inc., such as the Galaxy Tab from Samsung Electronics Co., Ltd. In an alternative embodiment, the programming computer is a personal digital assistant handheld computer operating under the Pocket-PC, Windows Mobile, Windows Phone, Windows RT, or Windows operating systems, licensed by Microsoft Corporation, Redmond, Washington, such as the Surface from Microsoft Corporation, the Dell Axim X5 and X50 personal data assistants, sold by Dell, Inc., Round Top, Tex., the HP Jornada personal data assistant, sold by Hewlett-Packard Company, Palo Alto, Tex. The programming computer 41 functions through those components conventionally found in such devices, including, for instance, a central processing unit, volatile and persistent memory, touch-sensitive display, control buttons, peripheral input and output ports, and network interface. The computer 41 operates under the control of the application software 45, which is executed as program code as a series of process or method modules or steps by the programmed computer hardware. Other assemblages or configurations of computer hardware, firmware, and software are possible.

Operationally, the programming computer 41, when connected to a neurostimulator 12 through wireless telemetry using the programming wand 42, can be used by a healthcare provider to remotely interrogate the neurostimulator 12 and modify stored stimulation parameters. The programming wand 42 provides data conversion between the digital data accepted by and output from the programming computer and the radio frequency signal format that is required for communication with the neurostimulator 12. In other embodiments, the programming computer may communicate with the implanted neurostimulator 12 using other wireless communication methods, such as wireless RF transmission. The programming computer 41 may further be configured to receive inputs, such as physiological signals received from patient sensors (e.g., implanted or external). These sensors may be configured to monitor one or more physiological signals, e.g., vital signs, such as body temperature, pulse rate, respiration rate, blood pressure, etc. These sensors may be coupled directly to the programming computer 41 or may be coupled to another instrument or computing device which receives the sensor input and transmits the input to the programming computer 41. The programming computer 41 may monitor, record, and/or respond to the physiological signals in order to effectuate stimulation delivery in accordance with embodiments of the present invention.

The healthcare provider operates the programming computer 41 through a user interface that includes a set of input controls 43 and a visual display 44, which could be touch-sensitive, upon which to monitor progress, view downloaded telemetry and recorded physiology, and review and modify programmable stimulation parameters. The telemetry can include reports on device history that provide patient identifier, implant date, model number, serial number, magnet activations, total ON time, total operating time, manufacturing date, and device settings and stimulation statistics and on device diagnostics that include patient identifier, model identifier, serial number, firmware build number, implant date, communication status, output current status, measured current delivered, lead impedance, and battery status. Other kinds of telemetry or telemetry reports are possible.

During interrogation, the programming wand 42 is held by its handle 46 and the bottom surface 47 of the programming wand 42 is placed on the patient's chest over the location of the implanted neurostimulator 12. A set of indicator lights 49 can assist with proper positioning of the wand and a set of input controls 48 enable the programming wand 42 to be operated directly, rather than requiring the healthcare provider to awkwardly coordinate physical wand manipulation with control inputs via the programming computer 41. The sending of programming instructions and receipt of telemetry information occur wirelessly through radio frequency signal interfacing. Other programming computer and programming wand operations are possible.

FIG. 4 is a diagram showing the helical electrodes 14 provided on the stimulation lead assembly 13 of FIG. 2 in place on a vagus nerve 15, 16 in situ 50. Although described with reference to a specific manner and orientation of implantation, the specific surgical approach and implantation site selection particulars may vary, depending upon physician discretion and patient physical structure.

Under one embodiment, helical electrodes 14 may be positioned on the patient's vagus nerve 61 oriented with the end of the helical electrodes 14 facing the patient's head. At the distal end, the insulated electrical lead body 13 is bifurcated into a pair of lead bodies 57, 58 that are connected to a pair of electrodes 51, 52. The polarity of the electrodes 51, 52 could be configured into a proximal anode and a distal cathode, or a proximal cathode and a distal anode. In addition, an anchor tether 53 is fastened over the lead bodies 57, 58 that maintains the helical electrodes' position on the vagus nerve 61 following implant. In one embodiment, the conductors of the electrodes 51, 52 are manufactured using a platinum and iridium alloy, while the helical materials of the electrodes 51, 52 and the anchor tether 53 are a silicone elastomer.

During surgery, the electrodes 51, 52 and the anchor tether 53 are coiled around the vagus nerve 61 proximal to the patient's head, each with the assistance of a pair of sutures 54, 55, 56, made of polyester or other suitable material, which help the surgeon to spread apart the respective helices. The lead bodies 57, 58 of the electrodes 51, 52 are oriented distal to the patient's head and aligned parallel to each other and to the vagus nerve 61. A strain relief bend 60 can be formed on the distal end with the insulated electrical lead body 13 aligned, for example, parallel to the helical electrodes 14 and attached to the adjacent fascia by a plurality of tie-downs 59a-b.

The neurostimulator 12 delivers VNS under control of the electronic circuitry 22. The stored stimulation parameters are programmable. Each stimulation parameter can be independently programmed to define the characteristics of the cycles of therapeutic stimulation and inhibition to ensure optimal stimulation for a patient 10. The programmable stimulation parameters include output current, signal frequency, pulse width, signal ON time, signal OFF time, magnet activation (for VNS specifically triggered by “magnet mode”), and reset parameters. Other programmable parameters are possible. In addition, sets or “profiles” of preselected stimulation parameters can be provided to physicians with the external programmer and fine-tuned to a patient's physiological requirements prior to being programmed into the neurostimulator 12, such as described in commonly-assigned U.S. Pat. No. 8,630,709, entitled “Computer-Implemented System and Method for Selecting Therapy Profiles of Electrical Stimulation of Cervical Vagus Nerves for Treatment of Chronic Cardiac Dysfunction,” the disclosure of which is incorporated by reference.

Therapeutically, the VNS may be delivered as a multimodal set of therapeutic doses, which are system output behaviors that are pre-specified within the neurostimulator 12 through the stored stimulation parameters and timing cycles implemented in firmware and executed by the microprocessor controller. The therapeutic doses include a maintenance dose that includes continuously-cycling, intermittent and periodic cycles of electrical stimulation during periods in which the pulse amplitude is greater than 0 mA (“therapy ON”) and during periods in which the pulse amplitude is 0 mA (“therapy OFF”).

The neurostimulator 12 can operate either with or without an integrated heart rate sensor, such as respectively described in commonly-assigned U.S. Pat. No. 8,577,458, entitled “Implantable Device for Providing Electrical Stimulation of Cervical Vagus Nerves for Treatment of Chronic Cardiac Dysfunction with Leadless Heart Rate Monitoring,” and U.S. patent application, entitled “Implantable Device for Providing Electrical Stimulation of Cervical Vagus Nerves for Treatment of Chronic Cardiac Dysfunction,” Ser. No. 13/314,119, filed on Dec. 7, 2011, pending, the disclosures of which are hereby incorporated by reference herein in their entirety. Additionally, where an integrated leadless heart rate monitor is available, the neurostimulator 12 can provide autonomic cardiovascular drive evaluation and self-controlled titration, such as respectively described in commonly-assigned U.S. Pat. No. 8,918,190, entitled “Implantable Device for Evaluating Autonomic Cardiovascular Drive in a Patient Suffering from Chronic Cardiac Dysfunction,” filed on Dec. 7, 2011, and U.S. Pat. No. 8,918,191, entitled “Implantable Device for Providing Electrical Stimulation of Cervical Vagus Nerves for Treatment of Chronic Cardiac Dysfunction with Bounded Titration,” filed on Dec. 7, 2011, the disclosures of which are incorporated by reference. Finally, the neurostimulator 12 can be used to counter natural circadian sympathetic surge upon awakening and manage the risk of cardiac arrhythmias during or attendant to sleep, particularly sleep apneic episodes, such as respectively described in commonly-assigned U.S. Pat. No. 8,923,964, entitled “Implantable Neurostimulator-Implemented Method For Enhancing Heart Failure Patient Awakening Through Vagus Nerve Stimulation,” filed on Nov. 9, 2012, the disclosure of which is incorporated by reference.

The VNS stimulation signal may be delivered as a therapy in a maintenance dose having an intensity that is insufficient to elicit undesirable side effects, such as cardiac arrhythmias. The VNS can be delivered with a periodic duty cycle in the range of 2% to 89% with a preferred range of around 4% to 36% that is delivered as a low intensity maintenance dose. Alternatively, the low intensity maintenance dose may comprise a narrow range approximately at 17.5%, such as around 15% to 25%. The selection of duty cycle is a tradeoff among competing medical considerations. The duty cycle is determined by dividing the stimulation ON time by the sum of the ON and OFF times of the neurostimulator 12 during a single ON-OFF cycle. However, the stimulation time may also need to include ramp-up time and ramp-down time, where the stimulation frequency exceeds a minimum threshold (as further described infra with reference to FIG. 7).

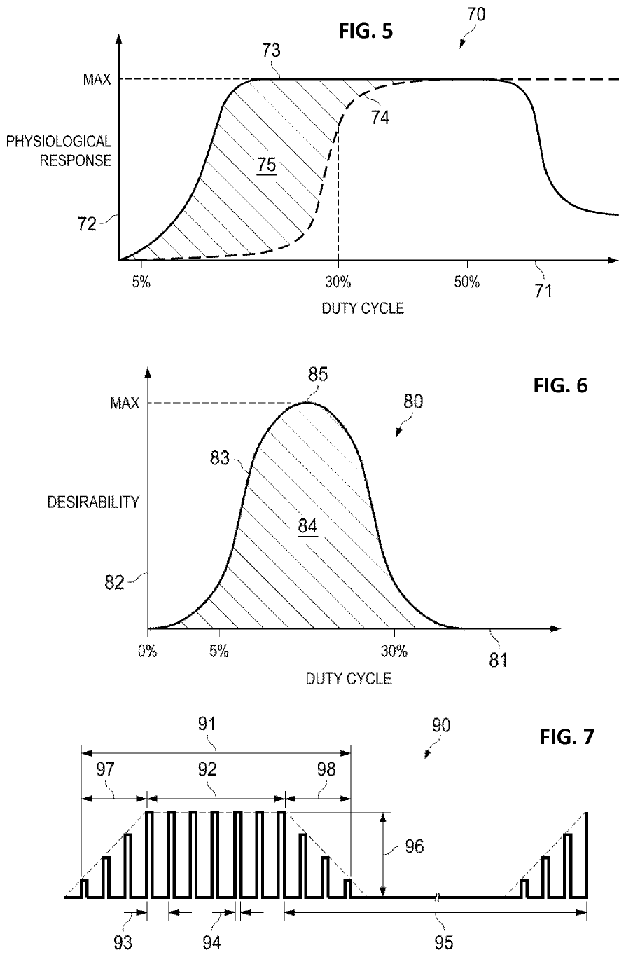

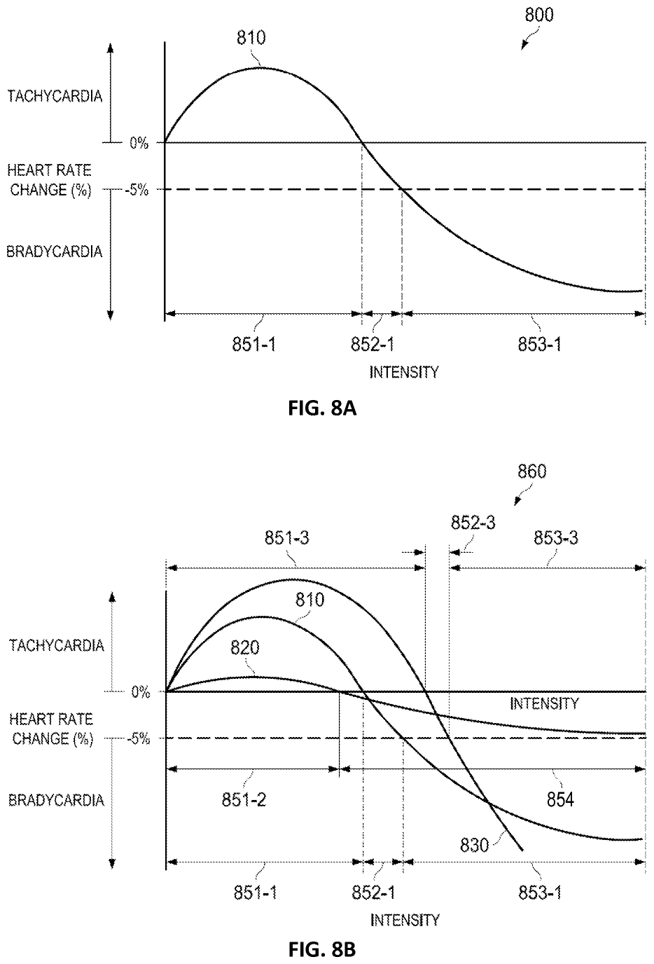

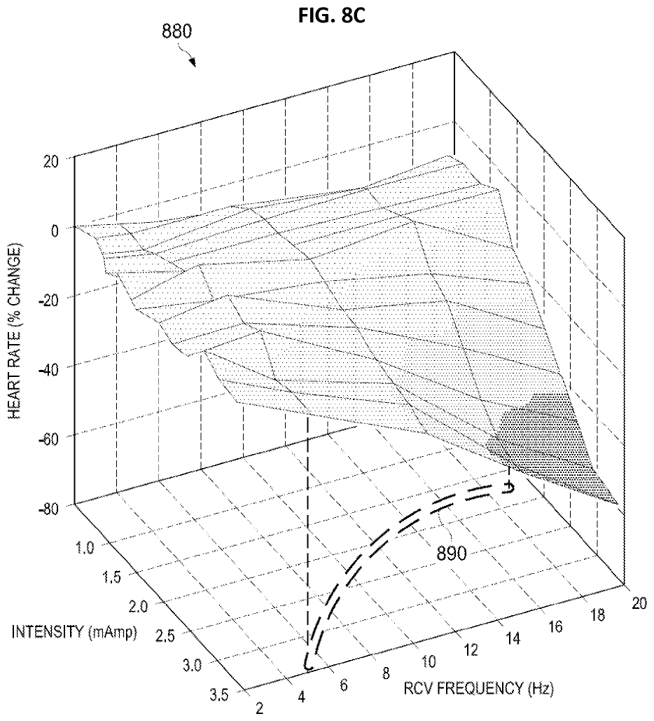

FIG. 5 is a graph 70 showing, by way of example, the relationship between the targeted therapeutic efficacy 73 and the extent of potential side effects 74 resulting from use of the implantable neurostimulator 12 of FIG. 1, after the patient has completed the titration process. The graph in FIG. 5 provides an illustration of the failure of increased stimulation intensity to provide additional therapeutic benefit, once the stimulation parameters have reached the neural fulcrum zone, as will be described in greater detail below with respect to FIG. 8. As shown in FIG. 5, the x-axis represents the duty cycle 71. The duty cycle is determined by dividing the stimulation ON time by the sum of the ON and OFF times of the neurostimulator 12 during a single ON-OFF cycle. However, the stimulation time may also include ramp-up time and ramp-down time, where the stimulation frequency exceeds a minimum threshold (as further described infra with reference to FIG. 7). When including the ramp-up and ramp-down times, the total duty cycle may be calculated as the ON time plus the ramp-up and ramp-down times divided by the OFF time, ON time, and ramp-up and ramp-down times, and may be, e.g., between 15% and 30%, and more specifically approximately 23%. The y-axis represents physiological response 72 to VNS therapy. The physiological response 72 can be expressed quantitatively for a given duty cycle 71 as a function of the targeted therapeutic efficacy 73 and the extent of potential side effects 74, as described infra. The maximum level of physiological response 72 (“max”) signifies the highest point of targeted therapeutic efficacy 73 or potential side effects 74.

Targeted therapeutic efficacy 73 and the extent of potential side effects 74 can be expressed as functions of duty cycle 71 and physiological response 72. The targeted therapeutic efficacy 73 represents the intended effectiveness of VNS in provoking a beneficial physiological response for a given duty cycle and can be quantified by assigning values to the various acute and chronic factors that contribute to the physiological response 72 of the patient 10 due to the delivery of therapeutic VNS. Acute factors that contribute to the targeted therapeutic efficacy 73 include beneficial changes in heart rate variability and increased coronary flow, reduction in cardiac workload through vasodilation, and improvement in left ventricular relaxation. Chronic factors that contribute to the targeted therapeutic efficacy 73 include improved cardiovascular regulatory function, as well as decreased negative cytokine production, increased baroreflex sensitivity, increased respiratory gas exchange efficiency, favorable gene expression, renin-angiotensin-aldosterone system down-regulation, anti-arrhythmic, anti-apoptotic, and ectopy-reducing anti-inflammatory effects. These contributing factors can be combined in any manner to express the relative level of targeted therapeutic efficacy 73, including weighting particular effects more heavily than others or applying statistical or numeric functions based directly on or derived from observed physiological changes. Empirically, targeted therapeutic efficacy 73 steeply increases beginning at around a 5% duty cycle, and levels off in a plateau near the maximum level of physiological response at around a 30% duty cycle. Thereafter, targeted therapeutic efficacy 73 begins decreasing at around a 50% duty cycle and continues in a plateau near a 25% physiological response through the maximum 100% duty cycle.

The intersection 75 of the targeted therapeutic efficacy 73 and the extent of potential side effects 74 represents one optimal duty cycle range for VNS. FIG. 6 is a graph 80 showing, by way of example, the optimal duty cycle range 83 based on the intersection 75 depicted in FIG. 5. The x-axis represents the duty cycle 81 as a percentage of stimulation time over stimulation time plus inhibition time. The y-axis represents therapeutic points 82 reached in operating the neurostimulator 12 at a given duty cycle 81. The optimal duty cycle range 83 is a function 84 of the intersection 75 of the targeted therapeutic efficacy 73 and the extent of potential side effects 74. The therapeutic operating points 82 can be expressed quantitatively for a given duty cycle 81 as a function of the values of the targeted therapeutic efficacy 73 and the extent of potential side effects 74 at the given duty cycle shown in the graph 70 of FIG. 5. The optimal therapeutic operating point 85 (“max”) signifies a tradeoff that occurs at the point of highest targeted therapeutic efficacy 73 in light of lowest potential side effects 74 and that point will typically be found within the range of a 5% to 30% duty cycle 81. Other expressions of duty cycles and related factors are possible.

Therapeutically and in the absence of patient physiology of possible medical concern, such as cardiac arrhythmias, VNS is delivered in a low level maintenance dose that uses alternating cycles of stimuli application (ON) and stimuli inhibition (OFF) that are tuned to activate both afferent and efferent pathways. Stimulation results in parasympathetic activation and sympathetic inhibition, both through centrally-mediated pathways and through efferent activation of preganglionic neurons and local circuit neurons. FIG. 7 is a timing diagram showing, by way of example, a stimulation cycle and an inhibition cycle of VNS 90, as provided by implantable neurostimulator 12 of FIG. 1. The stimulation parameters enable the electrical stimulation pulse output by the neurostimulator 12 to be varied by both amplitude (output current 96) and duration (pulse width 94). The number of output pulses delivered per second determines the signal frequency 93. In one embodiment, a pulse width in the range of 100 to 250 psec delivers between 0.02 mA and 50 mA of output current at a signal frequency of about 10 Hz, although other therapeutic values could be used as appropriate. In general, the stimulation signal delivered to the patient may be defined by a stimulation parameter set comprising at least an amplitude, a frequency, a pulse width, and a duty cycle.

In one embodiment, the stimulation time is considered the time period during which the neurostimulator 12 is ON and delivering pulses of stimulation, and the OFF time is considered the time period occurring in-between stimulation times during which the neurostimulator 12 is OFF and inhibited from delivering stimulation or configured to deliver a negligible or ineffective stimulation.

In another embodiment, as shown in FIG. 7, the neurostimulator 12 implements a stimulation time 91 comprising an ON time 92, a ramp-up time 97 and a ramp-down time 98 that respectively precede and follow the ON time 92. Under this embodiment, the ON time 92 is considered to be a time during which the neurostimulator 12 is ON and delivering pulses of stimulation at the full output current 96. Under this embodiment, the OFF time 95 is considered to comprise the ramp-up time 97 and ramp-down time 98, which are used when the stimulation frequency is at least 10 Hz, although other minimum thresholds could be used, and both ramp-up and ramp-down times 97, 98 last two seconds, although other time periods could also be used. The ramp-up time 97 and ramp-down time 98 allow the strength of the output current of each output pulse to be gradually increased and decreased, thereby avoiding deleterious reflex behavior due to sudden delivery or inhibition of stimulation at a programmed intensity corresponding to the full output current 96.

Therapeutic vagus neural stimulation has been shown to provide cardioprotective effects. Although delivered in a maintenance dose having an intensity that is insufficient to elicit undesirable side effects, such as cardiac arrhythmias, ataxia, coughing, hoarseness, throat irritation, voice alteration, or dyspnea, therapeutic VNS can nevertheless potentially ameliorate pathological tachyarrhythmias in some patients. Although VNS has been shown to decrease defibrillation threshold, VNS has not been shown to terminate VF in the absence of defibrillation. VNS prolongs ventricular action potential duration, so may be effective in terminating VT. In addition, the effect of VNS on the AV node may be beneficial in patients with AF by slowing conduction to the ventricles and controlling ventricular rate.

### Neural Fulcrum Zone

As described above, autonomic regulation therapy results in simultaneous creation of action potentials that simultaneously propagate away from the stimulation site in afferent and efferent directions within axons comprising the cervical vagus nerve complex. Upon stimulation of the cervical vagus nerve, action potentials propagate away from the stimulation site in two directions, efferently toward the heart and afferently toward the brain. Different parameter settings for the neurostimulator 12 may be adjusted to deliver varying stimulation intensities to the patient. The various stimulation parameter settings for current VNS devices include output current amplitude, signal frequency, pulse width, signal ON time, and signal OFF time.

When delivering neurostimulation therapies to patients, it is generally desirable to avoid stimulation intensities that result in either excessive tachycardia or excessive bradycardia. However, researchers have typically utilized the patient's heart rate changes as a functional response indicator or surrogate for effective recruitment of nerve fibers and engagement of the autonomic nervous system elements responsible for regulation of heart rate, which may be indicative of therapeutic levels of VNS. Some researchers have proposed that heart rate reduction caused by VNS stimulation is itself beneficial to the patient.

In accordance with some embodiments, a neural fulcrum zone is identified, and neurostimulation therapy is delivered within the neural fulcrum zone. This neural fulcrum zone corresponds to a combination of stimulation parameters at which autonomic engagement is achieved but for which a functional response determined by heart rate change is nullified due to the competing effects of afferently and efferently-transmitted action potentials. In this way, the tachycardia-inducing stimulation effects are offset by the bradycardia-inducing effects, thereby minimizing side effects such as significant heart rate changes while providing a therapeutic level of stimulation. One method of identifying the neural fulcrum zone is by delivering a plurality of stimulation signals at a fixed frequency but with one or more other parameter settings changed so as to gradually increase the intensity of the stimulation.

FIGS. 8A-8C provide illustrative charts reflecting the location of the neural fulcrum zone. FIG. 8A is a chart 800 illustrating a heart rate response in response to such a gradually increased intensity at a first frequency, in accordance with embodiments of the present invention. In this chart 800, the x-axis represents the intensity level of the stimulation signal, and the y-axis represents the observed heart rate change from the patient's baseline basal heart rate observed when no stimulation is delivered. In this example, the stimulation intensity is increased by increasing the output current amplitude.

A first set 810 of stimulation signals is delivered at a first frequency (e.g., 10 Hz). Initially, as the intensity (e.g., output current amplitude) is increased, a tachycardia zone 851-1 is observed, during which period, the patient experiences a mild tachycardia. As the intensity continues to be increased for subsequent stimulation signals, the patient's heart rate response begins to decrease and eventually enters a bradycardia zone 853-1, in which a bradycardia response is observed in response to the stimulation signals. As described above, the neural fulcrum zone is a range of stimulation parameters at which the functional effects from afferent activation are balanced with or nullified by the functional effects from efferent activation to avoid extreme heart rate changes while providing therapeutic levels of stimulation. In accordance with some embodiments, the neural fulcrum zone 852-1 can be located by identifying the zone in which the patient's response to stimulation produces either no heart rate change or a mildly decreased heart rate change (e.g., <5% decrease, or a target number of beats per minute). As the intensity of stimulation is further increased at the fixed first frequency, the patient enters an undesirable bradycardia zone 853-1. In these embodiments, the patient's heart rate response is used as an indicator of autonomic engagement. In other embodiments, other physiological responses may be used to indicate the zone of autonomic engagement at which the propagation of efferent and afferent action potentials are balanced to identify the neural fulcrum zone.

FIG. 8B is a chart 860 illustrating a heart rate response in response to such a gradually increased intensity at two additional frequencies, in accordance with embodiments of the present invention. In this chart 860, the x-axis and y-axis represent the intensity level of the stimulation signal and the observed heart rate change, respectively, as in FIG. 8A, and the first set 810 of stimulation signals from FIG. 8A is also shown.

A second set 820 of stimulation signals is delivered at a second frequency lower than the first frequency (e.g., 5 Hz). Initially, as the intensity (e.g., output current amplitude) is increased, a tachycardia zone 851-2 is observed, during which period, the patient experiences a mild tachycardia. As the intensity continues to be increased for subsequent stimulation signals, the patient's heart rate response begins to decrease and eventually enters a mild heart rate reduction zone 854, in which a mild decrease in heart rate is observed in response to the stimulation signals. The low frequency of the stimulation signal in the second set 820 of stimulation signals limits the functional effects of nerve fiber recruitment and, as a result, the heart response remains relatively limited. Although this low frequency stimulation results in minimal heart rate reduction, and, therefore, minimal side effects, the stimulation intensity is too low to result in effective recruitment of nerve fibers and engagement of the autonomic nervous system. As a result, a therapeutic level of stimulation is not delivered.

A third set of 830 of stimulation signals is delivered at a third frequency higher than the first and second frequencies (e.g., 20 Hz). As with the first set 810 and second set 820, at lower intensities, the patient first experiences a tachycardia zone 851-3. At this higher frequency, the level of increased heart rate is undesirable. As the intensity is further increased, the heart rate decreases, similar to the decrease at the first and second frequencies but at a much higher rate. The patient first enters the neural fulcrum zone 852-3 and then the undesirable bradycardia zone 853-3. Because the slope of the curve for the third set 830 is much steeper than the first set 810, the region in which the patient's heart rate response is between 0% and −5% (e.g., the neural fulcrum zone 852-3) is much narrower than the neural fulcrum zone 852-1 for the first set 810. Accordingly, when testing different operational parameter settings for a patient by increasing the output current amplitude by incremental steps, it can be more difficult to locate a programmable output current amplitude that falls within the neural fulcrum zone 852-3. When the slope of the heart rate response curve is high, the resulting heart rate may overshoot the neural fulcrum zone and create a situation in which the functional response transitions from the tachycardia zone 851-3 to the undesirable bradycardia zone 853-3 in a single step. At that point, the clinician would need to reduce the amplitude by a smaller increment or reduce the stimulation frequency in order to produce the desired heart rate response for the neural fulcrum zone 852-3.

FIG. 8C is a chart 880 illustrating mean heart rate response surfaces in conscious, normal dogs during 14 second periods of right cervical vagus VNS stimulation ON-time. The heart rate responses shown in z-axis represent the percentage heart rate change from the baseline heart rate at various sets of VNS parameters with the pulse width set at 250 psec, the pulse amplitude ranging from 0 mA to 3.5 mA (provided by the x-axis), and the pulse frequency ranging from 2 Hz to 20 Hz (provided by the y-axis). Curve 890 roughly represents the range of stimulation amplitude and frequency parameters at which a null response (i.e., 0% heart rate change from baseline) is produced. This null response curve 890 is characterized by the opposition of functional responses (e.g., tachycardia and bradycardia) arising from afferent and efferent activation.

### Titration Process

Several classes of implantable medical devices provide therapy using electrical current as a stimulation vehicle. When such a system stimulates certain organs or body structures like the vagus nerve, therapeutic levels of electrical stimulation are usually not well tolerated by patients without undergoing a process known as titration. Titration is a systematic method of slowly increasing, over time, stimulation parameters employed by an implanted device to deliver stimulation current until therapeutic levels become tolerated by the patient.

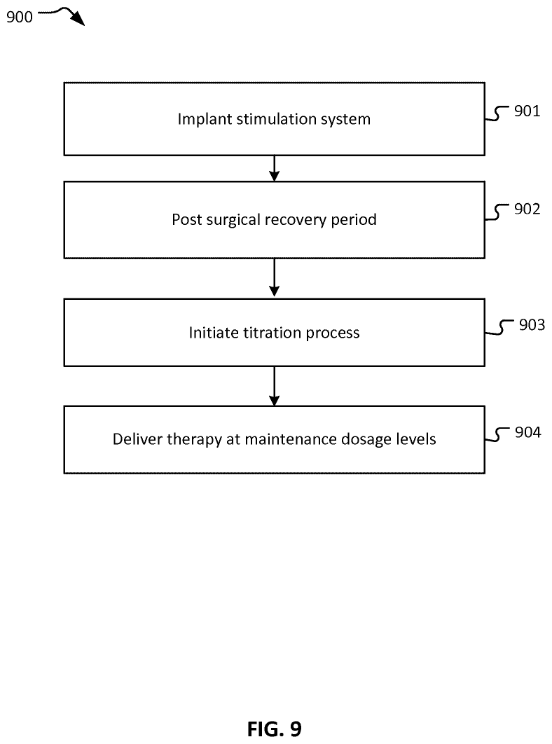

FIG. 9 is a flow diagram showing a method for delivering vagus nerve stimulation therapy 900, in accordance with embodiments of the present invention. A titration process is used to gradually increase the stimulation intensity to a desired therapeutic level. If the stimulation intensity is increased too quickly before the patient is fully accommodated to the stimulation signal, the patient may experience undesirable side effects, such as coughing, hoarseness, throat irritation, or expiratory reflex. The titration process gradually increases stimulation intensity within a tolerable level, and maintains that intensity for a period of time to permit the patient to adjust to each increase in intensity, thereby gradually increasing the patient's side effect tolerance zone boundary to so as to accommodate subsequent increases in intensity. The titration process continues until adequate adaptation is achieved. In preferred embodiments, the titration process is automated and is executed by the implanted device without manual adjustment of the stimulation intensity by the subject or health care provider. As will be described in greater detail below, adequate adaptation is a composite threshold comprising one or more of the following: an acceptable side effect level, a target intensity level, and a target physiological response. In preferred embodiments, adequate adaption includes all three objectives: an acceptable side effect level, a target intensity level, and a target physiological response.

As described above, it may be desirable to minimize the amount of time required to complete the titration process so as to begin delivery of the stimulation at therapeutically desirable levels, particularly when the patient is being treated for an urgent condition such as CHF. In addition, it is desirable to utilize a maintenance dose intensity at the minimum level required to achieve the desired therapeutic effect. This can reduce power requirements for the neurostimulator and reduce patient discomfort.

It has been observed that a patient's side effect profile is more sensitive to the stimulation output current than to the other stimulation parameters, such as frequency, pulse width, and duty cycle. As a result, accommodation to the stimulation output current is a primary factor in completing the titration process. It has also been observed that if the other stimulation parameters are maintained at a level below the target levels, the output current can be increased to higher levels without eliciting undesirable side effects that would be result when the other parameters are at the target level. As a result, increasing the target output current while maintaining the other stimulation parameters (pulse width in particular) at reduced levels can result in a faster accommodation and shorter overall titration time than would be achieved by attempting to increase the output current while stimulating at the target pulse width.

In step 901, a stimulation system 11, including a neurostimulator 12, a nerve stimulation lead assembly 13, and a pair of electrodes 14, is implanted in the patient. In step 902, the patient undergoes an optional post-surgery recovery period, during which time the surgical incisions are allowed to heal and no VNS therapy occurs. This period may last, e.g., two weeks post surgery. In step 903, the stimulation therapy process is initiated. During this process, VNS therapy is titrated by adjusting one or more of the stimulation parameters, including output current, pulse width, signal frequency, and duty cycle, as will be described in greater detail below. Completion of the titration process determines the stimulation intensity to be used for subsequent maintenance doses delivered in step 904. These maintenance doses may be selected to provide the minimum stimulation intensity necessary to provide the desired therapeutic result.

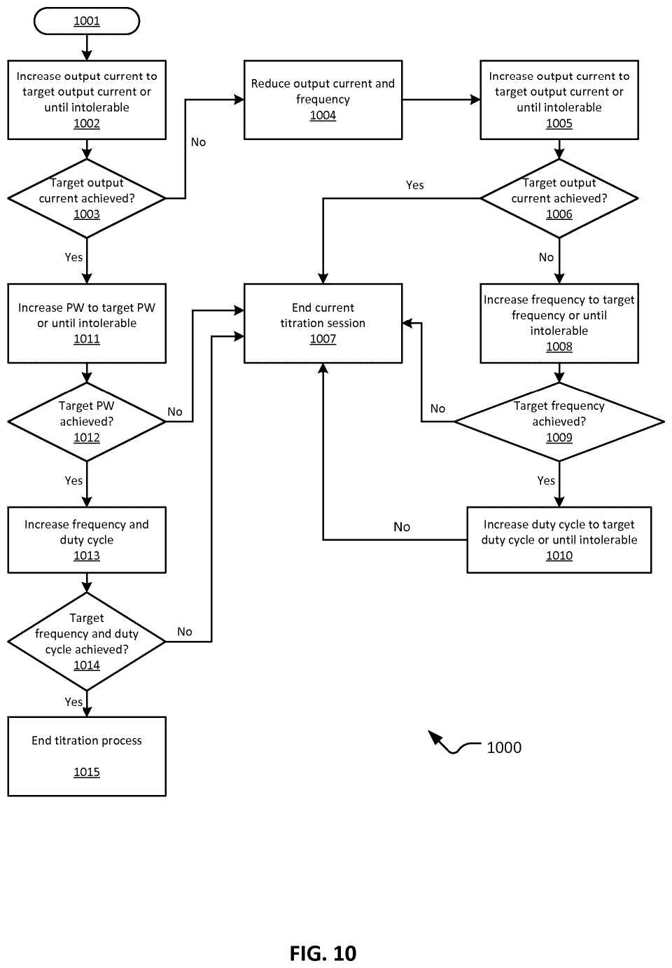

FIG. 10 is a flow diagram illustrating a titration process 1000 in accordance with embodiments of the present invention. When first initiating the titration process, the neurostimulator 11 is configured to generate a stimulation signal having an initial stimulation parameter set. The initial parameter set may comprise an initial output current, an initial frequency, an initial pulse width, and an initial duty cycle. The various initial parameter settings may vary, but may be selected so that one or more of the parameters are set at levels below a predefined target parameter set level, such that the titration process is used to gradually increase the intensity parameters to achieve adequate adaptation. In some embodiments, the initial frequency is set at the target frequency level, while the initial output current, initial pulse width, and initial duty cycle are set below their respective target levels. In one embodiment, the target parameter set comprises a 10 Hz frequency, 250 μsec pulse width, a duty cycle of 14 sec ON and 1.1 minutes OFF, and an output current of between 1.5 mA-3.0 mA (e.g., 2.5 mA for right side stimulation and 3.0 mA for left side stimulation), and the initial parameter set comprises 10 Hz frequency, 130 μsec pulse width, a duty cycle of 14 sec ON and 1.1 minutes OFF, and an output current of between 0.25 mA-0.5 mA. In other embodiments, the target parameter set includes a 5 Hz frequency is used instead of a 10 Hz frequency.

In step 1001, the stimulation system delivers stimulation to the patient. If this is the first titration session, then the stimulation would be delivered with the initial stimulation parameter set described above. If this is a subsequent titration session, then the stimulation intensity would remain at the same level at the conclusion of the previous titration session.

In step 1002, the output current is gradually increased until the stimulation results in an intolerable side effect level, the target output current (e.g., 2.5 mA) is reached, or adequate adaptation is achieved. As described above, adequate adaptation is a composite threshold comprising one or more of the following: an acceptable side effect level, a target intensity level, and a target physiological response. In accordance with some embodiments, the target physiological response comprises a target heart rate change during stimulation. The patient's heart rate may be monitored using an implanted or external heart rate monitor, and the patient's heart rate during stimulation is compared to the patient's baseline heart rate to determine the extent of heart rate change. In accordance with some embodiments, the target heart rate change is a heart rate change of between 4% and 5%. If at any point during the titration process 1000 adequate adaptation is achieved, the titration process ends and the stimulation intensity which resulted in the adequate adaptation is used for ongoing maintenance dose therapy delivery.

The output current may be increased in any desired increment, but small increments, e.g., 0.1 mA or 0.25 mA, may be desirable so as to enable more precise adjustments. In some cases, the output current increments may be determined by the neurostimulator's maximum control capability. During the initial titration sessions, it is likely that the patient's side effect tolerance zone boundary will be reached well before the output current reaches the target level or adequate adaptation is achieved. At decision step 1003, if the target output current has not been achieved but the maximum tolerable side effects have been exceeded, the process proceeds to step 1004.

In step 1004, the output current is reduced one increment to bring the side effects within acceptable levels. In addition, the frequency is reduced. In embodiments in which the initial frequency was 10 Hz, in step 1004, the frequency may be reduced, e.g., to 5 Hz or 2 Hz.

Next, in step 1005, the output current is gradually increased again at the reduced frequency level until the stimulation results in an intolerable side effect level or the target output current (e.g., 2.5 mA) is reached. At decision step 1006, if the target output current has not been reached but the maximum tolerable side effects have been exceeded, the process proceeds to step 1007.

In step 1007, the titration session is concluded. The stimulation system may be programmed to continue delivering the stimulation signal at the last parameter settings achieved prior to conclusion of the titration session. After a period of time, another titration session may be initiated and the process returns to step 1001. This can be any period of time sufficient to permit the patient to adjust to the increased stimulation levels. This can be, for example, as little as approximately two or three days, approximately one to two weeks, approximately four to eight weeks, or any other desired period of time.

In some embodiments, the titration sessions are automatically initiated by the stimulation system or initiated by the patient without requiring any intervention by the health care provider. This can eliminate the need for the patient to schedule a subsequent visit to the health care provider, thereby potentially reducing the total amount of time needed for the titration process to complete. In these embodiments, the stimulation system includes a physiological monitor, e.g., an implanted heart rate sensor, that communicates with the stimulation system's control system to enable the control system to detect when the target physiological response has been achieved and conclude the titration process. The stimulation system could in addition or alternatively include a patient control input to permit the patient to communicate to the control system that the acceptable side effect level has been exceeded. This control input may comprise an external control magnet that the patient can swipe over the implanted neurostimulator, or other internal or external communication device that the patient can use to provide an input to the control system. In these automatically initiated titration sessions, the stimulation system may be configured to wait a period of time after completing one session before initiating the next session. This period of time may be predetermined, e.g., two or three days.

Returning to decision step 1006, if the target output current has not been reached but the maximum tolerable side effects have been exceeded, the process proceeds to step 1008. In step 1008, the output current is reduced one increment to restore an acceptable side effect condition, and the frequency is gradually increased until the stimulation results in an intolerable side effect level or the target frequency (e.g., 10 Hz) is reached. At decision step 1009, if the target frequency has not been reached but the maximum tolerable side effects have been exceeded, the frequency is reduced to restore an acceptable side effect level and the process proceeds to step 1007. Again, in step 1007, the current titration session is concluded and the stimulation system may be programmed to continue delivering the stimulation signal at the last parameter settings achieved prior to conclusion of the titration session.

At decision step 1009, if the target frequency has been reached before the maximum tolerable side effects have been exceeded, the process proceeds to step 1010 and the duty cycle is gradually increased until the stimulation results in an intolerable side effect level or the target duty cycle (e.g., 14 sec ON and 1.1 min OFF) is reached, at which point the process proceeds to step 1007 and the titration session is concluded and ongoing stimulation delivered at the last intensity eliciting acceptable side effect levels.

Returning to decision step 1003, if the target output current has been achieved before the maximum tolerable side effects are exceeded, the process proceeds to step 1011. In step 1011, the pulse width is gradually increased until the stimulation results in an intolerable side effect level or the target pulse width (e.g., 250 μsec) is reached. In some embodiments, before step 1011, the output current is reduced (e.g., by up to 50%), and the pulse width may be increased in step 1011 at that reduced output current. After the target pulse width is achieved in step 1012, the output current may be restored to the target output current. In other embodiments, the output current may be reduced (or may be retained at the reduced level established prior to step 1011, as described above), and the frequency and duty cycle are gradually increased in step 1013 (described below) at that reduced output current. This reduction in output current after achieving the target output current may enable the patient to maintain tolerability with increasing pulse width, frequency, and duty cycle in subsequent titration steps.

At decision step 1012, if the target pulse width has not been achieved before the maximum tolerable side effects have been exceeded, the pulse width is reduced to restore an acceptable side effect level and the process proceeds to step 1007. Again, in step 1007, the current titration session is concluded.

If at decision step 1012, the target pulse width has been achieved before the maximum tolerable side effects have been exceeded, the process proceeds to step 1013. In step 1013, the frequency and duty cycle are increased until the stimulation results in an intolerable side effect level or the target frequency and target duty cycle are reached. The frequency and duty cycle can be increased in step 1013 simultaneously, sequentially, or on an alternating basis.

At decision step 1014, if the target frequency and target duty cycle have not been achieved before the maximum tolerable side effects have been exceeded, the pulse width and/or frequency are reduced to restore an acceptable side effect level and the process continues to step 1007 and the titration session is concluded.

At decision step 1014, if the target pulse width and target frequency have been achieved before the maximum tolerable side effects have been exceeded, all of the stimulation parameters will have reached their target levels and the titration process concludes at step 1015. The stimulation therapy may proceed with the maintenance dose at the target stimulation levels.

In some embodiments, in step 1004, instead of reducing the frequency in order to facilitate increase of the output current, the pulse width may be reduced. For example, embodiments where the target pulse width is 250 μsec, the pulse width may be reduced, e.g., to 150 μsec or less. Then, the method proceeds to step 1005, in which the output current is gradually increased again at the reduced pulse width level until the stimulation results in an intolerable side effect level or the target output current (e.g., 2.5 mA) is reached.

Therapy can also be autonomously titrated by the neurostimulator 12 in which titration progressively occurs in a self-paced, self-monitored fashion. The progression of titration sessions may occur on an autonomous schedule or may be initiated upon receipt of an input from the patient. Ordinarily, the patient 10 is expected to visit his healthcare provider to have the stimulation parameters stored by the neurostimulator 12 in the recordable memory 29 reprogrammed using an external programmer. Alternatively, the neurostimulator 12 can be programmed to automatically titrate therapy by up titrating the VNS through periodic incremental increases as described above. The titration process 1000 will continue until the ultimate therapeutic goal is reached.

Following the titration period, therapeutic VNS, as parametrically defined by the maintenance dose operating mode, is delivered to at least one of the vagus nerves. The stimulation system 11 delivers electrical therapeutic stimulation to the cervical vagus nerve of a patient 10 in a manner that results in creation and propagation (in both afferent and efferent directions) of action potentials within neuronal fibers of either the left or right vagus nerve independent of cardiac cycle.

In a further embodiment, the sensed heart rate data can be used to analyze therapeutic efficacy and patient condition. For instance, statistics could be determined from the sensed heart rate, either onboard by the neurostimulator 12 or by an external device, such as a programming computer following telemetric data retrieval. The sensed heart rate data statistics can include determining a minimum heart rate over a stated time period, a maximum heart rate over a stated time period, an average heart rate over a stated time period, and a variability of heart rate over a stated period, where the stated period could be a minute, hour, day, week, month, or other selected time interval. Still other uses of the heart rate sensor 31 and the sensed heart rate data are possible.

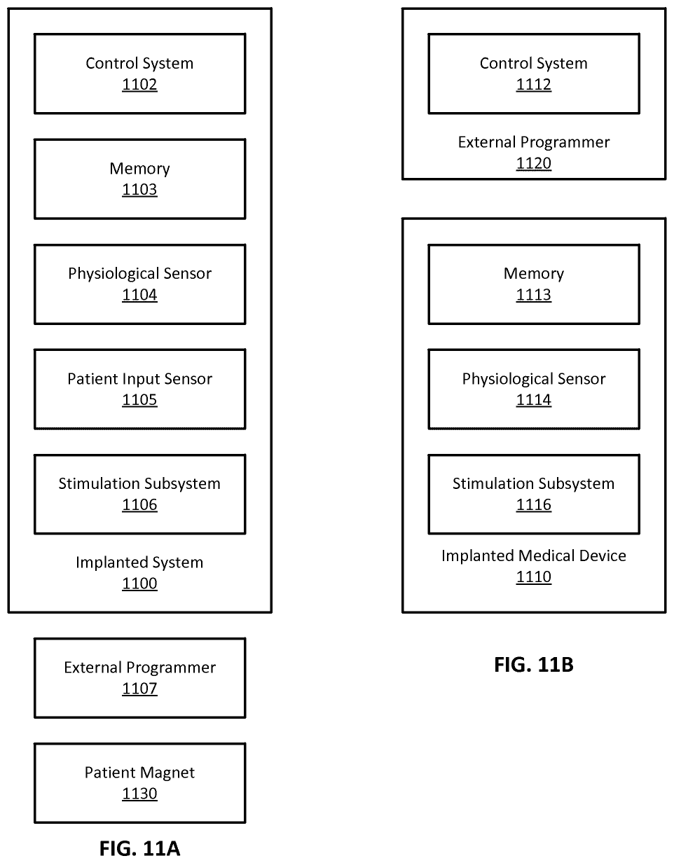

FIG. 11A is a simplified block diagram of an implanted neurostimulation system 1100 in accordance with embodiments of the present invention. The implanted neurostimulation system 1100 comprises a control system 1102 comprising a processor programmed to operate the system 1100, a memory 1103, an optional physiological sensor 1104, and a stimulation subsystem 1106. The physiological sensor 1104 may be configured to monitor any of a variety of patient physiological signals and the stimulation subsystem 1106 may be configured to deliver a stimulation signal to the patient. In one example, the physiological sensor 1104 comprises an ECG sensor for monitoring heart rate and the stimulation subsystem 1106 comprises a neurostimulator 12 programmed to deliver ON-OFF cycles of stimulation to the patient's vagus nerve.

The control system 1102 is programmed to activate the neurostimulator 12 to deliver varying stimulation intensities to the patient and to monitor the physiological signals in response to those stimulation signals.

The external programmer 1107 shown in FIG. 11A may be utilized by a clinician or by the patient for communicating with the implanted system 1100 to adjust parameters, activate therapy, retrieve data collected by the system 1100 or provide other input to the system 1100. In some embodiments, the external programmer 1107 may be configured to program the implanted system 1100 with a prescribed time or window of time during which titration sessions may be initiated. This can be used to prevent a titration session from occurring at night when the patient's sleep is likely to be disturbed by the increase in stimulation intensity and resulting side effects.

Patient inputs to the implanted system 1100 may be provided in a variety of ways. The implanted system 1100 may include a patient input sensor 1105. As described above, a patient magnet 1130 may be used to provide external input to the system 1100. When the patient magnet 1130 is placed on the patient's chest in close proximity to the implanted system 1100, the patient input sensor 1105 will detect the presence of the magnetic field generated by the patient magnet 1130 and provide a control input to the control system 1102. The system 1100 may be programmed to receive patient inputs to set the time of day during which titration sessions are to be initiated.

In other embodiments, the patient input sensor 1105 may comprise a motion sensor, such as an accelerometer, which is configured to detect tapping on the surface of the patient's chest. The patient may use finger taps in one or more predetermined patterns to provide control inputs to the implanted system 1100. For example, when the motion sensor detects three rapid taps to the patient's chest, that may trigger an operation on the implanted system 1100 (e.g., to initiate a titration session). Alternatively, if the motion sensor detects a predetermined pattern of taps during a titration session, the implanted system 1100 will interpret those taps as a patient input indicating that the patient's tolerance zone boundary has been exceeded.

In other embodiments, the patient input sensor 1105 may comprise an acoustic transducer or other sensor configured to detect acoustic signals. The system 1100 may be programmed to interpret the detection of certain sounds as patient inputs. For example, the patient may utilize an electronic device, such as a smartphone or other portable audio device, to generate one or more predetermined sequences of tones. The system 1100 may be programmed to interpret each of these sequences of tones as a different patient input.

In other embodiments, the patient input sensor 1105 may be configured to detect when a patient is coughing, which can be interpreted by the system 1100 as an indication that the increased stimulation intensity exceeds the patient's tolerance zone boundary. The coughing could be detected by an accelerometer to detect movement of the patient's chest, an acoustic transducer to detect the sound of the patient's coughing, or both.

The titration of the stimulation signal delivery and the monitoring of the patient's physiological response (e.g., heart rate) may be advantageously implemented using control system in communication with both the stimulation subsystem 1106 and the physiological sensor 1104, such as by incorporating all of these components into a single implantable device. In accordance with other embodiments, the control system may be implemented in a separate implanted device or in an external programmer 1120 or other external device, as shown in FIG. 11B. The external programmer 1120 in FIG. 11B may include a control system 1112 and be utilized by a clinician or by the patient for adjusting stimulation parameters. The external programmer 1120 is in wireless communication with the implanted medical device 1110, which includes the stimulation subsystem 1116. In the illustrated embodiment, the physiological sensor 1114 is incorporated into the implanted medical device 1110, but in other embodiments, the sensor 1114 may be incorporated into a separate implanted device, may be provided externally and in communication with the external programmer 1120, or may be provided as part of the external programmer 1120. The implanted medical device 1110 may include a memory 1113 that may store information in preparation for a transmission to the external programmer 1120.

### Personalized Titration Via Parametric Modification

Titration is a method of varying over time stimulation parameters employed by an implanted device to deliver stimulation current, until therapeutic levels become tolerated by the patient. Embodiments provided above describe automated titration processes used to gradually increase the stimulation intensity to a desired therapeutic level. During periodic titration sessions, the stimulation intensity is increased until the maximum tolerable side effects are exceeded, at which point the stimulation intensity is reduced to a tolerable level and the patient is provided with a period of time to adapt to the new intensity levels before the next titration session is initiated. In some embodiments, the titration sessions may occur on a regular schedule (e.g., every two weeks), with an acclimation interval in between each titration session during which time stimulation at a tolerable intensity level is delivered. However, patients adapt to increased stimulation intensity levels at different rates, so the minimum acclimation interval required before the next titration session can successfully be initiated varies. In other embodiments, parameters other than or in addition to the acclimation interval may be adjusted based on the actual adaption experienced by the patient. The parameters that might be adjusted include, for example: current amplitude, pulse width, frequency, and OFF time.

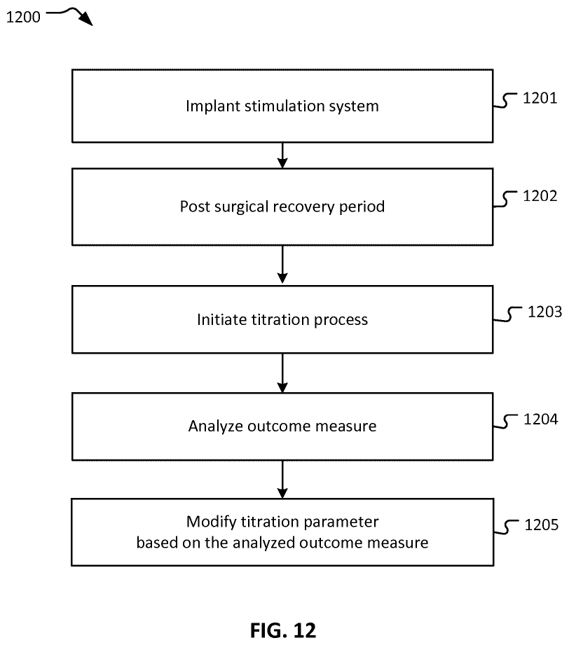

In accordance with some embodiments of the present invention, an automated titration process is provided which utilizes an acclimation interval between titration sessions that may be adjusted based on the patient's response to the stimulation. FIG. 12 illustrates a titration process 1200 with a variable acclimation interval. Steps 1201-1203 are similar to steps 901-903 illustrated in FIG. 9 and described above. However, in step 1204, an outcome measure for the titration sessions is analyzed. In step 1205, the acclimation interval between subsequent titration sessions is adjusted based on the analyzed outcome measure. If the outcome measure indicates that the patient is adapting to the stimulation at a slower than expected rate, then the acclimation interval may be increased to provide the patient with additional time to recover and adapt to each set of increased stimulation intensities. Conversely, if the outcome measure indicates that the patient is adapting to the stimulation at a faster than expected rate, then the acclimation interval may be decreased to accelerate the adaption process and reduce the overall time required to complete the titration process and achieve a tolerable therapeutic maintenance dose level.

Any of a variety of outcome measures may be used. In some embodiments, the outcome measure is the patient's tolerance of a targeted increase in one or more of the stimulation parameters. For example, if the patient is unable to tolerate any increase in stimulation output current (or stimulation parameter) over the course of two or more titration sessions separated by a default acclimation interval (e.g., two weeks), it may be concluded that the patient is adapting to the stimulation at a slower than expected rate. In response, the acclimation interval between subsequent titration sessions may be increased (to, e.g., three or more weeks). If the patient continues to be incapable of tolerating any increase in stimulation output current in subsequent titration sessions, then the acclimation interval may be increased again (to, e.g., four or more weeks).

In some cases, the patient may initially adapt to the increased stimulation intensity at a slower than expected rate, but after the acclimation interval is increased and subsequent titration sessions are successful at achieving the desired outcome measure, the patient's adaptation may accelerate, thereby permitting reduction of the acclimation interval back to the initial interval length. Accordingly, if the patient begins to adapt to the titration sessions after an increase in the acclimation interval, the system 1100 may be programmed to gradually reduce the acclimation interval in subsequent titration sessions.

In various embodiments described above, after a titration session is terminated, the system may be programmed to continue delivering stimulation at the last parameter settings achieved prior to conclusion of the titration session at an intensity just below the patient's tolerance zone boundary. This stimulation is delivered at this constant intensity until the next titration session is initiated. In some cases, patients are capable of enduring stimulation intensities just past the tolerance zone boundary for limited periods of time. The intensity levels just past the tolerance zone boundary may be considered by the patient as “moderately tolerable.” Patients may be willing to endure stimulation at the moderately tolerable levels for limited periods of time if it results in acceleration of the adaption process.

In accordance with some embodiments, after a titration session is concluded or at any desired periodicity during the acclimation interval, an elevated stimulation session may be initiated, during which time stimulation at moderately tolerable levels exceeding the tolerance zone boundary is delivered. This elevated stimulation session may continue for any desired period of time, such as, e.g., several minutes or several hours, after which point the stimulation intensity will be reduced to a sustained stimulation intensity level below the tolerance zone boundary. In some embodiments, the elevated stimulation session may continue for less than one day, while the sustained stimulation is delivered for a period greater than one day, or the elevated stimulation session may continue for less than six hours, while the sustained stimulation is delivered for a period greater than one week. Any desired periods of time may be used.

### Interactive Training Sessions

Various methods are described herein for titrating stimulation by gradually increasing stimulation intensity until the patient's tolerance zone boundary is reached or exceeded. In accordance with embodiments of the present invention, systems and methods are provided for performing interactive training sessions in clinic for patients about to undergo titration on an ambulatory basis. The methods permit clinicians to create a series of stimulation intensities (ranging from un-noticeable to noticeable but tolerable to intolerable), the patient's response to each stimulation, and the implanted device's response to patient inputs.

The implanted medical device 1100 may be used in conjunction with an external clinician programmer 1107 and patient input device (e.g., patient magnet 1130 or wireless-communications-enabled patient control device), to perform the titration processes on an ambulatory basis as described above, but is also programmed to execute in a training mode. This training mode may be initiated by the clinician using the clinician programmer 1107 while the patient is physically in the clinic for treatment and training. The training mode may be similar to the titration sessions described above, except that the increasing stimulation is initiated by the clinician using the programmer 1107 or automatically on an accelerated schedule. When the stimulation intensity reaches the patient's tolerance zone boundary, the patient can use any of the herein described methods for providing a patient input to the device 1100 to indicate that the tolerance zone boundary has been reached. When in training mode, the device 1100 may also transmit to the clinician programmer 1107 information regarding the stimulation being delivered. The programmer 1107 may include a display which permits the clinician to observe the increasing intensity and receive a report of the intensity level that elicited the patient input indicating that the tolerance zone boundary was reached. The display on the programmer 1107 may also be used to display feedback or instructions to the patient.

The clinician may run the training mode multiple times so that the patient may become proficient at recognizing stimulation levels that are noticeable but tolerable, and distinguishing those tolerable levels from the truly intolerable stimulation levels. This can also provide training for the patient in the proper use of the patient input device. In some embodiments, the programmer 1107 may be used to select the stimulation parameter to be increased (e.g., output current, frequency, pulse width, or duty cycle), so that the patient and clinician can observe the different responses that may be elicited depending on the parameter being adjusted. In some embodiments, the programmer 1107 may be configured to pause the titration algorithm to hold the stimulation at a single level. This may be useful for facilitating a tolerance zone assessment by providing the patient additional time to experience the stimulation. The programmer 1107 may also be used to terminate the training mode and return the device 1100 to its normal ambulatory mode, during which the desired ambulatory titration process may be performed.

The training mode may also comprise an algorithm that sequences stimulation changes based on the training mode parameters programmed by the clinician. Stimulation may be altered on a highly accelerated time scale in order to move the patient from tolerable to noticeable-but-tolerable to intolerable stimulation levels within the normal office follow-up period. This accelerated time scale may be, for example, five, ten or fifteen minutes for all training. This is in contrast to the ambulatory mode titration process that seeks to advance therapy levels without the patient exceeding the tolerance-zone boundary. Having the patient experience all three tolerance phases in a single clinic visit can provide valuable patient training, resulting in accelerated adaptation speed.

The system 1100 may be programmed with an autonomous monitor to ensure that the training mode terminates automatically after a certain period has elapsed, even in the absence of a termination input from the clinician programmer. For example, the system 1100 may be programmed to automatically time-out and terminate the training mode 24 hours after initiation. After this automatic time-out, the system 1100 may automatically initiate the ambulatory mode.

As a result, the system may enable patients to experience stimulation levels (usually following a stimulation increase) that may be unacceptable. Patients may also learn how to effectively deal with the intolerance through the use of the external patient input device. Clinicians can learn how individual patients react to various stimulation levels and the patients' cognitive ability to deal with unacceptable stimulation autonomously. Clinicians may also gain a sense of stimulation increases that an individual patient can tolerate and adjust the ambulatory titration algorithm accordingly.

### T-Wave Alternans Target

T-wave alternans (TWA) is a periodic beat-to-beat variation in the amplitude or shape of the T-wave of a subject's ECG. TWA is a marker of cardiac electrical instability, and has been used for arrhythmia risk stratification. Experiments have indicated that TWA is fundamentally linked to vulnerability to ventricular fibrillation (VF) and is an established market of risk for sudden cardiac death (SCD) in patients with diverse cardiovascular diseases, including heart failure. In accordance with embodiments of the present invention, TWA may be used in place of or in addition to the heart rate monitoring described above in connection with the titration of stimulation and determination of optimal stimulation parameters for autonomic regulation therapy (ART). Analysis of TWA in heart failure patients receiving ART has shown a relationship between stimulation current amplitude and TWA.

As previously described, when delivering neurostimulation therapies to patients, it is generally desirable to avoid stimulation intensities that result in either excessive tachycardia or excessive bradycardia. In various embodiments above, a patient's heart rate response or a target stimulation parameter such as output current may be utilized when titrating stimulation and determining optimal stimulation parameters. In other embodiments, a patient's TWA may be used instead of or in addition to the heart rate response and target output current during stimulation titration. An implantable ART system which includes a sensor for monitoring cardiac rhythm may be used. The implanted device can calculate TWA at various points during the titration period. This TWA calculation may be performed using methods similar to published algorithms, as will be described in greater detail below. The system could first establish a TWA baseline value monitored during the post-implant period (e.g. 1-2 weeks after implantation) before the initiation of stimulation. The titration process may then be automatically performed by the implanted device, but instead of titrating to a target output current, as described above, the device would titrate stimulation intensity until a target TWA change from the established baseline TWA or target TWA level is observed. Once that target TWA change is achieved, the titration process is completed and the device may continue delivering the autonomic regulation therapy at the stimulation parameter settings that resulted in the target TWA change. In some embodiments, after the titration is complete and the therapeutic levels of stimulation are delivered for some period of time, the device may periodically monitor TWA levels and adjust stimulation as needed to maintain the target TWA change or target TWA level.

Various methods may be used for determining the patient's TWA. One approach is to utilize a modified moving average analysis, as described in Nearing et al., “Modified Moving Average Analysis of T-Wave Alternans to Predict Ventricular Fibrillation with High Accuracy,” J. Appl. Physiol. 92: 541-549 (2002), the disclosure of which is incorporated by reference herein in its entirety. This approach involves constructing modified moving average computed beats by averaging alternate ECG beats. A weighted moving average is applied to limit the contribution of any one beat. The alternans estimate for any ECG segment is then determined as the maximum difference between A and B modified moving average computed beats within the ST segment and T-wave region. Another approach is to utilize a spectral analysis method of analyzing TWA in the frequency domain, as described in Smith et al., “Electrical alternans and cardiac electrical instability,” Circulation 77, No. 1, 110-121 (1988), the disclosure of which is incorporated by reference herein in its entirety. This approach utilizes a spectral analysis procedure to quantify the degree and statistical significance of waveform alternation present in the magnitude of the three orthogonal-lead ECG.

The ECG signal may be obtained using a sensor provided in a variety of locations. In some embodiments, the ECG sensor is incorporated into the housing of the implanted neurostimulator, or the ECG may be detected using implanted ECG leads coupled to the implanted neurostimulator. In other embodiments, ECG may be monitored using an external device which is in communication with the implanted neurostimulator. The calculation of TWA and determination of change from the baseline TWA may be performed by either the external or implanted devices.

The target TWA change from the baseline TWA or target TWA value may be any value that corresponds with the desired therapeutic effect. In some embodiments, the target TWA change from the baseline TWA may be in the range of approximately −20 μV to approximately −30 μV, e.g., a target TWA decrease of 25 μV. In some embodiments, the target TWA change from the baseline TWA may be a decrease of at least 10 μV.

In one experiment, subjects were implanted with a VNS therapy system (DEMIPULSE Model 103 pulse generator and PERENNIAFLEX Model 304 lead, both from LivaNova PLC of Houston, Texas) with randomized lead placement on either the right (N=12) or left cervical vagus nerve (N=11). The pulse generator was activated at 15±3 days after implantation. All patients were initially stimulated at a pulse width of 130 μs and a pulse frequency of 10 Hz, continuous cyclic 14-sec active (ON) and 66-sec inactive (OFF) to produce a duty cycle of 18%; 1,080 cycles/day. Stimulation parameters were systematically adjusted during periodic clinic visits over a 10-week titration period to a pulse width of 250 μs, a pulse frequency of 10 Hz, and target output current amplitude of 1.5-3.0 mA. VNS activation and inactivation periods were unrelated to the cardiac cycle (i.e., open loop), so no intracardiac sensing lead was used. During titration sessions, VNS intensity was gradually increased in 0.25 mA steps with the use of a radiofrequency programmer (Model 250 programming system from LivaNova PLC) to levels that produced acute VNS-related side effects (referred to as the “VNS tolerance zone boundary”), such as activation of the expiratory reflex (mild cough) or moderate heart rate reduction during the VNS-active phase. When the VNS tolerance zone boundary was established by evidence of expiratory reflex activation or heart rate reduction, the output current was reduced by ≥1 output current step (0.25 mA) to ensure that the therapy was well tolerated. During the 10-week titration period, the VNS stimulation intensity was progressively increased to an average output current of 2.0±0.1 mA (for left side implantation patients: 2.2±0.2 mA; for right side implantation patients: 1.8±0.2 mA). Continuous cyclic stimulation was maintained at a tolerable level throughout the titration period and the 12-month follow-up period. Effects of low intensity (<2 mA, n=9) and high intensity (≥2 mA, n=14) stimulation levels were compared.

Ambulatory ECG 24-hour recordings were made using an externally-worn Holter monitor at baseline and after six and twelve months of chronic therapy. Measurements of TWA, HRT, HRV, ventricular tachycardia (VT) incidence, and heart rate were performed by an investigator.

Peak TWA was quantified from standard precordial leads V₁ and V₅ and aVF in 24-hour recordings with the Modified Moving Average (MMA) method, as described in the Nearing publication cited above. Limb leads were not recorded, as they are prone to motion artifact associated with daily activities. The MMA method employs the noise-rejection principle of recursive averaging. The maximum TWA level throughout the recording is reported as the TWA value for that patient. As established in patients with heart disease, a TWA threshold level of greater than or equal to 47 μV was defined in this study as an abnormal TWA test, and greater than or equal to 60 μV was defined as markedly abnormal.

In the 23 patients that were followed up for twelve months, mean TWA was 70±4.9 μV at baseline (left side: 73±7.3 μV; right side: 68±8.4 μV). Peak TWA in the pooled cohort (n=23) did not improve after six months of continuous cyclic stimulation, but improved significantly after twelve months of therapy (by −20±4.5 μV, p=0.0002). At baseline, 83% (19 of 23) of patients had TWA of greater than or equal to 60 μV, the threshold for strongly abnormal levels. At six months, 78% (18 of 23) had TWA of greater than or equal to 60 μV. At twelve months, 65% (15 of 23) had TWA of greater than or equal to 60 μV. No patient achieved TWA less than 47 μV at baseline or during the follow up year. Improvement in TWA was similar with both left-sided and right-sided stimulation. Patients who were titrated to a low intensity of stimulation (less than 2.0 mA, n=9) did not show a significant improvement in TWA at either six months or twelve months. Patients who were titrated to a higher intensity of stimulation (greater than or equal to 2.0 mA, n=14) showed a significant improvement in TWA at twelve months (by −23±5.9 μV, p=0.002).

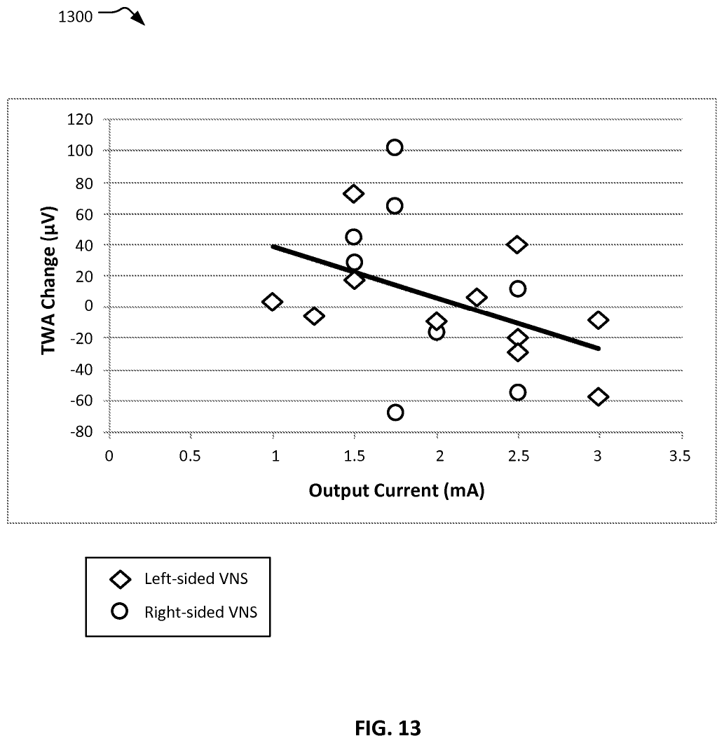

FIG. 13 is a scatter plot 1300 illustrating stimulation output current and corresponding T-wave alternans changes from baseline. The stimulation was delivered at a frequency of 10 Hz, a pulse width of 250-μsec, and a duty cycle of 18%. At stimulation output currents below 2.0 mA, all but one of the data points in plot 1300 are indicative of an undesirable increase in TWA from the baseline TWA. Accordingly, during the titration process, if an increase in TWA from the baseline TWA is observed, the device may be programmed to increase the output current (or other stimulation intensity parameter) until the target TWA change is achieved. At stimulation output currents of 2.0 mA and above, all but three of the data points in plot 1300 are indicative of a decrease in TWA from the baseline TWA. When a decrease in TWA from the baseline TWA is observed, it may be concluded that the stimulation intensity is achieving a positive therapeutic effect. The device may be programmed to terminate the titration process and begin delivering ongoing therapy at the stimulation intensity that produced the desired decrease in TWA from baseline.

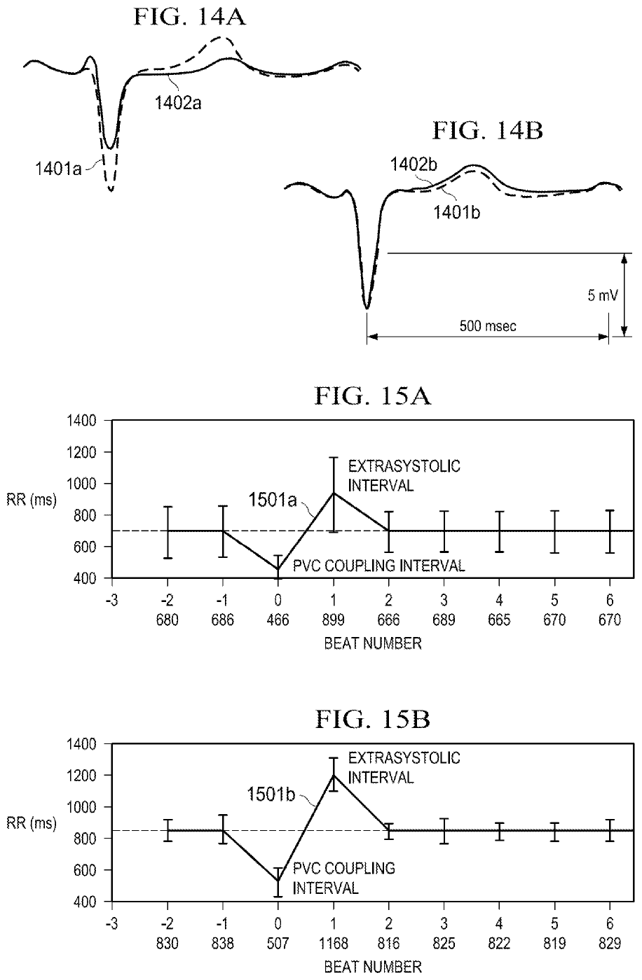

FIGS. 14A-14B are assessments of TWA using the MMA method in a single representative patient with heart failure. The patient's ECG recordings were separated into alternating A beats and B beats, and then an average was taken of each to produce an average A Beat signal 1401 and an average B Beat signal 1402. These signals were superimposed to determine the TWA. FIG. 14A illustrates the QRS-aligned superimposed A Beat 1401a and B Beat 1402a taken at baseline, resulting in a TWA of 97 μV, calculated by performing a time-dependent difference between the curves and calculating the maximum value in the T-wave period. This TWA level of 97 μV is greater than the 60 μV threshold for strongly abnormal levels. FIG. 14B illustrates the QRS-aligned superimposed A Beat 1401b and B Beat 1402b taken after twelve months of therapy, resulting in a TWA of 42 μV, which is less than the 47 μV threshold for abnormal levels. As can be seen in FIGS. 14A-14B, the patient's TWA levels experienced a significant decrease after twelve months of VNS therapy.

Heart rate turbulence (HRT) is an indicator of baroreflex sensitivity. In this experiment, various HRT parameters were measured for the patients at baseline and after twelve months of VNS therapy. The HRT parameter turbulence onset (TO) calculates the initial brief acceleration of sinus rate after a premature ventricular contraction (PVC). The HRT parameter turbulence slope (TS) characterizes the subsequent heart rate deceleration after the PVC.

FIGS. 15A-15B show HRT response to the VNS stimulation therapy over the twelve month period in a representative patient. FIG. 15A shows the R-R interval in msec (y-axis) as a function of time (x-axis, showing the beat number), taken at baseline, prior to implantation of the pulse generator. FIG. 15B shows the R-R interval taken after twelve months of VNS therapy. The TO taken at baseline was not statistically different than the TO taken at either six or twelve months. However, in FIG. 15A, the baseline HRT turbulence slope (TS) 1501a was 5.02 msec, and in FIG. 15B, the HRT TS 1501b had nearly doubled to 9.71 msec. This increase is indicative of significantly improved baroreceptor sensitivity.

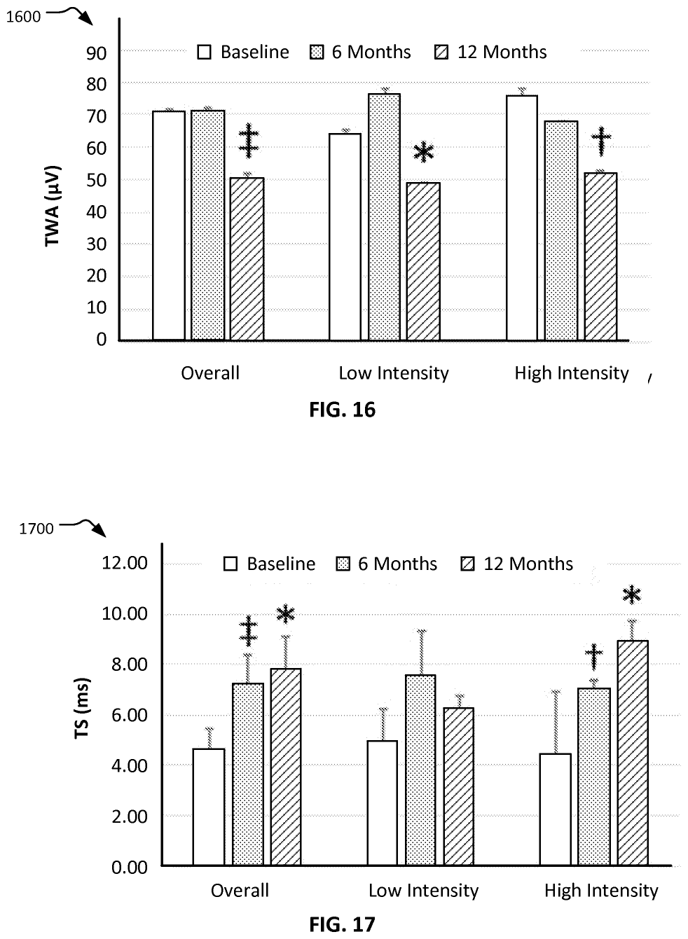

FIG. 16 is a bar chart 1600 illustrating the TWA magnitude levels (in μV) taken at baseline, at six months, and at twelve months for subjects receiving low intensity stimulation (pulse amplitude less than 2.0 mA), subjects receiving high intensity stimulation (pulse amplitude greater than or equal to 2.0 mA), and overall for all subjects. In the fifteen patients receiving high intensity VNS, TWA was reduced from 75.6±5.7 to 51.8±2.5 μV at twelve months (†p<0.001). In the ten patients receiving low intensity VNS, TWA was reduced from 64.0±0.5 to 48.5±2.5 μV at twelve months (*p<0.05). For all of the 25 patients, TWA was reduced from 71.0±4.6 to 50.5±1.8 μV at twelve months (‡p<0.0001). As illustrated in FIG. 16, the change in TWA levels was significant for all patients.

FIG. 17 is a bar chart 1700 illustrating the heart rate turbulence slope (TS) levels (in msec) taken at baseline, at six months, and at twelve months for subjects receiving low intensity stimulation (pulse amplitude less than 2.0 mA), subjects receiving high intensity stimulation (pulse amplitude greater than or equal to 2.0 mA), and overall for all subjects. In the fifteen patients receiving high intensity VNS, there was a significant increase in TS at six months (from 4.4±2.5 to 7.0±0.3 msec, †p<0.005) with a further increase at twelve months (to 8.9±0.8 msec, *p<0.025). In the overall cohort of 25 patients, there were increases in TS at six months (from 4.6±0.8 to 7.2±1.2 msec, ‡p<0.001) with a further increase at twelve months (to 7.8±1.3 msec, *p<0.025). Notably, the low intensity subjects did not experience a statistically significant change in their TS levels. This suggests that only high intensity stimulation can produce improvement in TS, and is therefore more efficacious in treating heart failure patients.

As a result of these experiments, it is believed that chronic, high intensity VNS stimulation for autonomic regulation therapy in patients with symptomatic heart failure can decrease cardiac electrical instability, as reflected in reduced TWA levels and suppression of VT, and can improve baroreflex sensitivity, as reflected in increased HRT slope, both indicators of cardiovascular mortality risk. However, there appears to be an intensity-related effect indicative of a dose-response relationship of VNS, underscoring the importance of appropriate VNS parameter selection to optimize the potential benefits of ART. This method of chronic VNS may provide a safe and effective means not only to improve cardiac mechanical function in patients with depressed left ventricular ejection fraction (LVEF) but also to protect against life-threatening ventricular arrhythmias and to improve cardioprotective autonomic reflexes.

In accordance with embodiments of the present invention, TWA and/or TS may be utilized when titrating stimulation and determining optimal stimulation parameters. During titration, the stimulation intensity may be increased until a target TWA magnitude or target TWA change from baseline is detected. The targets may vary, but some possible target TWA magnitudes include, for example, 45 μV, 47 μV, or 50 μV, and some possible target TWA changes from baseline include, for example, 5 μV, 7.5 μV, 10 μV, 12.5 μV, or 15 μV. Alternatively, the stimulation intensity may be increased until a target TS magnitudes or target TS change from baseline is detected. The targets may vary, but some possible target TS magnitudes include, for example, 1.0 msec, 2.5 msec, or 5.0 msec, and some possible target TS changes from baseline include, for example, increases of 25%, 50%, 75%, 100%, 125%, or 150%.

As described above, the heart rate measurements used to determine TWA and/or TS levels may be made by the implanted device using an implanted ECG heart rate sensor. In other embodiments, the heart rate measurements may be made using an external heart rate sensor. This sensor may be in communication with the implanted device or an external device, which can calculate the TWA and/or TS levels based on the ECG signals obtained by the external heart rate sensor.

In accordance with various embodiments, the frequency with which the subject's heart rate is measured and the TWA and/or TS levels are determined may vary. In some embodiments, the heart rate is measured and TWA and/or TS levels calculated on a substantially continuous basis during the titration process and optionally after titration is completed in order to determine whether subsequent intensity changes may be desired. In other embodiments, the heart rate measurement and TWA and/or TS level calculations may be performed on a periodic basis, such as e.g., daily, weekly, monthly, semi-annually, or annually. This may be performed only during the initial titration process or also after titration has been completed. In some embodiments, multiple measurements can be made daily or weekly and used to generate a more robust average daily or weekly value. In other embodiments, the heart rate measurements and TWA and/or TS level calculations may be made on demand by the subject's health care provider. For example, the health care provider may transmit a control input to the implanted device to cause the implanted device to begin a period of heart rate monitoring and/or TWA/TS calculations.

Other variations of methods of utilizing TWA monitoring to deliver stimulation therapy may be used. In embodiments described herein, the TWA baseline value is established either prior to implantation or in the post-implant period before initiation of stimulation. In other embodiments, after delivery of VNS therapy for an extended period of time, the stimulation may be suspended for some period of time and a new TWA baseline value determined. It may be desirable to reset the TWA baseline on a periodic basis, such as, e.g., after the patient has received one month, several months, one year, or several years of autonomic regulation therapy.

In other embodiments, instead of a target TWA change from baseline TWA, the target may be an absolute value of TWA magnitude, rather than a relative change from baseline. For example, if the magnitude of the observed TWA is above a threshold level (e.g., 65 μV), then the device would increase stimulation intensity until the TWA decreases to an acceptable level.

In embodiments described above, the neurostimulation system provides an implantable device providing closed-loop therapy, wherein adjustments to the stimulation intensity are made based on TWA, TS, and/or TO calculations from cardiac signals monitored by the implanted device. In other embodiments, the cardiac signals may be made by a different implanted device or an external device in communication with the implanted stimulator. In yet other embodiments, the neurostimulation system could be open loop without making automatic changes to the stimulation intensity. In these embodiments, the cardiac signals may be made by the implanted device or by an external device, and the TWA, TS, and/or TO calculations based on those cardiac signals may be made by the implanted device or by an external device. Information about the TWA, TS, and/or TO calculations (e.g., magnitudes, changes from baseline, historical measurements, etc.) could be communicated to the patient and/or health care provider using, for example, an external device. The patient and/or health care provider can use this information to make adjustments to the stimulation therapy, to make adjustments to other therapies, such as medication adjustments, or merely to monitor the patient's progress.

In accordance with embodiments of the invention, examples are provided below:

Example 1. A method of operating an implantable medical device (IMD) comprising a neurostimulator coupled to an electrode assembly, said neurostimulator adapted to deliver a stimulation signal to a patient, said method comprising: monitoring an electrocardiogram (ECG) of the patient; activating the IMD to deliver to the patient a plurality of stimulation signals at increasing stimulation intensities until a target threshold is detected, wherein the target threshold comprises one or more of a target T-wave alternans (TWA) threshold, a target heart rate turbulence slope (TS) threshold, and a target heart rate turbulence onset (TO) threshold; and activating the IMD to deliver a therapeutic level of stimulation corresponding to the stimulation intensity at which the target threshold was detected.

Example 2. The method according to Example 1, wherein: the target threshold comprises the target TWA threshold.

Example 3. The method according to Example 2, wherein: the target TWA threshold comprises a TWA less than a target TWA magnitude.

Example 4. The method according to Example 3, wherein: the target TWA magnitude comprises approximately 47 μV.

Example 5. The method according to Example 2, further comprising: computationally determining a baseline TWA for the patient; wherein the target TWA threshold comprises a change in TWA from the baseline TWA equal to or greater than a target TWA change.

Example 6. The method according to Example 5, wherein: the target TWA change comprises approximately 10 μV.

Example 7. The method according to Example 5, further comprising: performing a baseline update process, the baseline update process comprising: temporarily ceasing the delivery of stimulation to the patient; monitoring the ECG of the patient to determine an updated baseline TWA for the patient; activating the IMD to deliver to the patient a plurality of stimulation signals at increasing stimulation intensities until a target change in TWA from the updated baseline TWA is observed; and activating the IMD to deliver a therapeutic level of stimulation corresponding to the stimulation intensity during which the target change in TWA from the updated baseline TWA was observed.

Example 8. A method of operating an implantable medical device (IMD) comprising a neurostimulator coupled to an electrode assembly, said neurostimulator adapted to deliver a stimulation signal to a patient, said method comprising: monitoring an electrocardiogram (ECG) of the patient to determine a baseline for the patient, wherein the baseline comprises one or more of a baseline T-wave alternans (TWA), a baseline heart rate turbulence slope (TS), and a baseline heart rate turbulence onset (TO); activating the IMD to deliver to the patient a plurality of stimulation signals at increasing stimulation intensities until a target change from the baseline is observed; and identifying a neural fulcrum zone corresponding to the stimulation intensity during which the target change from the baseline was observed.

Example 9. The method according to Example 8, wherein: the baseline comprises the baseline TWA; and the target change from the baseline comprises a target change in TWA from the baseline TWA.

Example 10. The method according to Example 8, further comprising: activating the IMD to chronically deliver stimulation signals in the identified neural fulcrum zone.

Example 11. The method according to Example 10, wherein said activating the IMD to chronically deliver stimulation signals in the identified neural fulcrum zone comprises activating the IMD to treat chronic cardiac dysfunction.

Example 12. The method according to Example 8, wherein: said activating the IMD to deliver to the patient the plurality of stimulation signals at increasing stimulation intensities comprises activating the IMD to deliver to the patient the plurality of stimulation signals at increasing stimulation output currents until a target change from the baseline is observed.

Example 13. The method according to Example 8, wherein: said IMD is adapted to deliver the stimulation signal to a vagus nerve of the patient.

Example 14. The method according to Example 8, further comprising: performing a baseline update process, the baseline update process comprising: temporarily ceasing the delivery of stimulation to the patient; monitoring the ECG of the patient to determine an updated baseline for the patient; activating the IMD to deliver to the patient a plurality of stimulation signals at increasing stimulation intensities until a target change from the updated baseline is observed; and identifying an updated neural fulcrum zone corresponding to the stimulation intensity during which the target change from the updated baseline was observed.

Example 15. A method of operating an implantable medical device (IMD) comprising a neurostimulator coupled to an electrode assembly, said neurostimulator adapted to deliver a stimulation signal to a patient, said method comprising: computationally determining a cardiac magnitude value for the patient, wherein the cardiac magnitude value comprises one or more of a T-wave alternans (TWA) magnitude value, a heart rate turbulence slope (TS) magnitude value, and a heart rate turbulence onset (TO) magnitude value; activating the IMD to deliver to the patient a plurality of stimulation signals at increasing stimulation intensities until the determined cardiac magnitude value reaches to a target cardiac magnitude; and activating the IMD to deliver a therapeutic level of stimulation corresponding to the stimulation intensity during which the determined cardiac magnitude value reached the target cardiac magnitude.

Example 16. The method according to Example 15, wherein: the computationally determining the cardiac magnitude value for the patient comprises computationally determining the TWA magnitude value; the activating the IMD to deliver to the patient the plurality of stimulation signals at increasing stimulation intensities comprises activating the IMD to deliver to the patient the plurality of stimulation signals at increasing stimulation intensities until the determined TWA magnitude value reaches to a target TWA magnitude; and the activating the IMD to deliver the therapeutic level of stimulation comprises activating the IMD to deliver the therapeutic level of stimulation corresponding to the stimulation intensity during which the determined TWA magnitude value reached the target TWA magnitude.

Example 17. The method according to Example 15, wherein: said computationally determining the cardiac magnitude value for the patient is performed by the IMD.

Example 18. A neurostimulation system, comprising: an implantable medical device (IMD) comprising: an electrode assembly; a neurostimulator coupled to the electrode assembly, wherein the neurostimulator is adapted to deliver a stimulation signal to a patient; and an electrocardiogram (ECG) sensor; and a control system. The control system being programmed to: monitor the ECG of the patient using the ECG sensor; activate the IMD to deliver to the patient a plurality of stimulation signals at increasing stimulation intensities until a target threshold is detected, wherein the target threshold comprises one or more of a target T-wave alternans (TWA) threshold, a target heart rate turbulence slope (TS) threshold, and a target heart rate turbulence onset (TO) threshold; and activate the IMD to deliver a therapeutic level of stimulation corresponding to the stimulation intensity at which the target threshold was detected.

Example 19. The system according to Example 18, wherein the target threshold comprises the target TWA threshold.

Example 20. The system according to Example 19, wherein the control system is programmed to: activate the IMD to deliver to the patient the plurality of stimulation signals at increasing stimulation intensities until a TWA less than a target TWA magnitude is detected.

Example 21. The system according to Example 20, wherein: the target TWA magnitude comprises approximately 47 μV.

Example 22. The system according to Example 18, wherein the control system is programmed to: computationally determine a baseline for the patient, wherein the baseline comprises one or more of a baseline TWA, a baseline TS, and a baseline TO; and activate the IMD to deliver to the patient the plurality of stimulation signals at increasing stimulation intensities until a change from the baseline equal to or greater than a target change is detected.

Example 23. The system according to Example 22, wherein: the baseline comprises the baseline TWA; the target change comprises a target TWA change of approximately 10 μV.

Example 24. The system according to Example 22, wherein the control system is programmed to: perform a baseline update process, the baseline update process comprising: temporarily ceasing the delivery of stimulation to the patient; monitoring the ECG of the patient to determine an updated baseline for the patient; activating the IMD to deliver to the patient a plurality of stimulation signals at increasing stimulation intensities until a target change from the updated baseline is observed; and activating the IMD to deliver a therapeutic level of stimulation corresponding to the stimulation intensity during which the target change from the updated baseline was observed.

Example 25. A neurostimulation system, comprising: an implantable medical device (IMD) comprising: an electrode assembly; a neurostimulator coupled to the electrode assembly, wherein the neurostimulator is adapted to deliver a stimulation signal to a patient; and an electrocardiogram (ECG) sensor; and a control system, wherein the control system is programmed to perform any of the methods described above in Examples 1-17.

Example 26. A neurostimulation system, comprising: an implantable medical device (IMD) comprising: an electrode assembly; a neurostimulator coupled to the electrode assembly, wherein the neurostimulator is adapted to deliver a stimulation signal to a patient; an electrocardiogram (ECG) sensor; and a control system. The control system being programmed to: activate the IMD to deliver to the patient a plurality of stimulation signals; monitor the ECG of the patient using the ECG sensor; and determine a cardiac value based on the monitored ECG, wherein the cardiac value comprises one or more of a T-wave alternans (TWA) value, a heart rate turbulence slope (TS) value, and a heart rate turbulence onset (TO) level.

While the invention has been particularly shown and described as referenced to the embodiments thereof, those skilled in the art will understand that the foregoing and other changes in form and detail may be made therein without departing from the spirit and scope. For example, in various embodiments described above, the stimulation is applied to the vagus nerve. Alternatively, spinal cord stimulation (SCS) may be used in place of or in addition to vagus nerve stimulation for the above-described therapies. SCS may utilize stimulating electrodes implanted in the epidural space, an electrical pulse generator implanted in the lower abdominal area or gluteal region, and conducting wires coupling the stimulating electrodes to the generator.

What is claimed is:

1. A method of operating an implantable medical device (IMD) comprising a neurostimulator coupled to an electrode assembly, said neurostimulator adapted to deliver a stimulation signal to a patient, the method comprising:
monitoring a T-wave alternans (TWA) magnitude value for the patient while delivering the stimulation signal to the patient;
transmitting the TWA magnitude value for the patient to an external device to be communicated to a health care provider; and
adjusting an intensity of the stimulation signal in accordance with an instruction provided via the external device by the health care provider until the TWA magnitude value reaches a target TWA magnitude value, the target TWA magnitude value corresponding to a desired therapeutic level.

2. The method of claim 1, wherein adjusting the intensity of the stimulation signal includes increasing the intensity of the stimulation signal.

3. The method of claim 1, further comprising:
delivering the stimulation signal at the intensity resulting in the target TWA magnitude value.

4. The method of claim 1, further comprising:
activating the IMD to deliver the stimulation signal to the patient.

5. The method of claim 1, further comprising:
determining that the TWA magnitude value is above a threshold value; and
upon determining that the TWA magnitude value is above the threshold value, increasing the intensity of the stimulation signal until a target TWA change is achieved.

6. The method of claim 1, further comprising:
determining a baseline TWA magnitude value prior to activating the IMD to deliver the stimulation signal;
increasing the intensity of the stimulation signal until a target TWA change between the baseline TWA magnitude value and the TWA magnitude value is reached, the target TWA change corresponding to the desired therapeutic level; and
delivering the stimulation signal at the intensity resulting in the target TWA change.

7. The method of claim 6, wherein the target TWA change from the baseline TWA magnitude value to the TWA magnitude value is a decrease of least one of 5 μV, 7.5 μV, 10 μV, 12.5 μV, 15 μV, 20 μV, or 30 μV.

8. The method of claim 6, wherein the target TWA magnitude value is at least one of 45 μV, 47 μV, or 50 μV.

9. The method of claim 1, wherein monitoring the TWA magnitude value comprises determining the TWA magnitude value on a continuous basis while delivering the stimulation signal to the patient.

10. The method of claim 1, wherein monitoring the TWA magnitude value comprises determining the TWA magnitude value on a periodic basis while delivering the stimulation signal to the patient.

11. The method of claim 1, wherein the TWA magnitude value is determined utilizing at least one of a modified moving average analysis or a spectral analysis.

12. The method of claim 1, further comprising transmitting historical TWA magnitude value measurements for the patient to the external device to be communicated to the health care provider.

13. A system, comprising:
a control system programmed to control a neurostimulator to deliver a stimulation signal to a patient, the control system programmed to:
monitor a T-wave alternans (TWA) magnitude value for the patient while delivering the stimulation signal to the patient;
transmit the TWA magnitude value for the patient to an external device to be communicated to a health care provider; and
adjust an intensity of the stimulation signal in accordance with an instruction provided via the external device by the health care provider until the TWA magnitude value reaches a target TWA magnitude value, the target TWA magnitude value corresponding to a desired therapeutic level.

14. The system of claim 13, wherein adjusting the intensity of the stimulation signal includes increasing the intensity of the stimulation signal.

15. The system of claim 13, wherein the control system is further programmed to:
deliver the stimulation signal at the intensity resulting in the target TWA magnitude value.

16. The system of claim 13, wherein the control system is further programmed to:
activate the neurostimulator to deliver the stimulation signal to the patient.

17. The system of claim 13, wherein the control system is further programmed to:
determine that the TWA magnitude value is above a threshold value; and
upon determining that the TWA magnitude value is above the threshold value, increase the intensity of the stimulation signal until a target TWA change is achieved.

18. A neurostimulation system, comprising:
an electrocardiogram (ECG) sensor; and
a control system programmed to control a neurostimulator to deliver a stimulation signal to a patient, the control system programmed to:
monitor a T-wave alternans (TWA) magnitude value for the patient while delivering the stimulation signal to the patient;
transmit the TWA magnitude value for the patient to an external device to be communicated to a health care provider; and
adjust an intensity of the stimulation signal in accordance with an instruction provided via the external device by the health care provider until the TWA magnitude value reaches a target TWA magnitude value, the target TWA magnitude value corresponding to a desired therapeutic level.

19. The neurostimulation system of claim 18, wherein adjusting the intensity of the stimulation signal includes increasing the intensity of the stimulation signal.

20. The neurostimulation system of claim 18, wherein the control system is further programmed to:
deliver the stimulation signal at the intensity resulting in the target TWA magnitude value.
